(19) United States
(12) Patent Application Publication
(10) Pub. No.: US 2006/0051353 A1
(43) Pub. Date: Mar. 9, 2006

(54) Methods and compositions to evaluate antibody treatment response

(76) Inventors: Jean-Frederic Colombel, La Madeleine (FR); Herve Watier, Ballan-Mire (FR); Edouard Louis, Liege (BE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/128,551

(22) Filed: May 13, 2005

Related U.S. Application Data
(60) Provisional application No. 60/571,407, filed on May 14, 2004.

(51) Int. Cl. A61K 39/395; C12Q 1/68; C12P 19/34

(52) U.S. Cl. 424/145.1; 435/6; 435/91.2

(57) ABSTRACT

The present invention relates to methods and compositions to evaluate or assess the response of a subject to particular therapeutic treatment. More particularly, the invention provides methods to determine the response of subjects, or to adapt the treatment protocol of subjects treated with therapeutic antibodies. The invention is based on a determination of the FCGR3A genotype of a subject. The invention can be used for patients with malignancies, particularly lymphoma, and is suited to select best responders and/or adjust treatment condition or protocol for low responders.

### CROSS REFERENCE TO RELATED APPLICATIONS

This application claims the benefit of U.S. Provisional Application Ser. No. 60/571,407, filed May 14, 2004, which is hereby incorporated by reference in its entirety, including all tables, references, figures and nucleic acid and polynucleotide sequence listings.

### FIELD OF THE INVENTION

The present invention relates to methods and compositions to evaluate or assess the response of a subject to particular therapeutic treatment. More particularly, the invention provides methods to determine the response of subjects, or to adapt the treatment protocol of subjects treated with therapeutic antibodies. The invention can be used for patients with malignancies, particularly lymphoma, and is suited to select best responders and/or adjust treatment condition or protocol for low responders.

### INTRODUCTION

Various therapeutic strategies in human beings are based on the use of therapeutic antibodies. This includes, for instance, the use of therapeutic antibodies developed to deplete target cells, particularly diseased cells such as virally-infected cells, tumor cells or other pathogenic cells, including allogenic immunocompetent cells. Such antibodies are typically monoclonal antibodies, of IgG species, typically IgG1 and IgG3. These antibodies can be recombinant antibodies and humanized antibodies, comprising functional domains from various species or origin or specificity. A particular example of such therapeutic antibodies is rituximab (Mabther®, Rituxan®), which is a chimeric anti-CD20 IgG1 monoclonal antibody made with human γ1 and κ constant regions linked to murine variable domains (Maloney D G, Liles T M, Czerwinski D K, et al.: Phase I clinical trial using escalating single-dose infusion of chimeric anti-CD20 monoclonal antibody (JDEC-C2B8) in patients with recurrent B-cell lymphoma. Blood. 1994;84:2457-2466). For a few years, rituximab has been considerably modifying the therapeutical strategy against B lymphoproliferative malignancies, particularly non-Hodgkin's lymphomas (NHL). Other examples of intact humanized IgG1 antibodies include alemtuzumab (Campath-1H), which is used in the treatment of B cell malignancies or trastuzumab (Herceptin®), which is used in the treatment of breast cancer. Additional examples of therapeutic antibodies under development are disclosed in the art.

While these antibodies represent a novel efficient approach to human therapy, particularly for treatment of tumors, they do not always exhibit a strong efficacy and their use could be improved by evaluating the response of subjects thereto. For instance, while rituximab, alone or in combination with chemotherapy was shown to be effective in the treatment of both low-intermediate (McLaughlin P, Grillo-Lopez A J, Link B K, et al.: Rituximab chimeric anti-CD20 monoclonal antibody therapy for relapsed indolent lymphoma: half of patients respond to a four-dose treatment program. J Clin Oncol. 1998;16:2825-2833; Maloney D G, Grillo-Lopez A J, White C A, et al.: IDEC-C2B8 (Rituximab) anti-CD20 monoclonal antibody therapy in patients with relapsed low-grade non-Hodgkin's lymphoma. Blood. 1997;90:2188-2195; Maloney D G, Grillo-Lopez A J, White C A, et al.: IDEC-C2B8 (Rituximab) anti-CD20 monoclonal antibody therapy in patients with relapsed low-grade non-Hodgkin's lymphoma. Blood. 1997;90:2188-2195; Hainsworth J D, Burris H A, 3rd, Morrissey L H, et al.: Rituximab monoclonal antibody as initial systemic therapy for patients with low-grade non-Hodgkin lymphoma. Blood. 2000;95:3052-3056; Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106; Coiffier B, Haloun C, Ketterer N, et al.: Rituximab (anti-CD20 monoclonal antibody) for the treatment of patients with relapsing or refractory aggressive lymphoma: a multicenter phase II study. Blood. 1998;92:1927-1932; Foran J M, Rohatiner A Z, Cunningham D, et al.: European phase II study of rituximab (chimeric anti-CD20 monoclonal antibody) for patients with newly diagnosed mantle-cell lymphoma and previously treated mantle-cell lymphoma, immunocytoma, and small B-cell lymphocytic lymphoma. J Clin Oncol. 2000;18:317-324; Anderson D R, Grillo-Lopez A, Varns C, Chambers K S, Hanna N: Targeted anti-cancer therapy using rituximab, a chimeric anti-CD20 antibody (IDEC-C2B8) in the treatment of non-Hodgkin's B-cell lymphoma. Biochem Soc Trans. 1997;25:705-708) and high-grade NHL(Coiffier B, Haioun C, Ketterer N, et al.: Rituximab (anti-CD20 monoclonal antibody) for the treatment of patients with relapsing or refractory aggressive lymphoma: a multicenter phase II study. Blood. 1998;92:1927-1932; Foran J M, Rohatiner A Z, Cunningham D, et al.: European phase II study of rituximab (chimeric anti-CD20 monoclonal antibody) for patients with newly diagnosed mantle-cell lymphoma and previously treated mantle-cell lymphoma, immunocytoma, and small B-cell lymphocytic lymphoma. J Clin Oncol. 2000;18:317-324; Anderson D R, Grillo-Lopez A, Tarns C, Chambers K S, Hanna N: Targeted anti-cancer therapy using rituximab, a chimeric anti-CD20 antibody (IDEC-C2B8) in the treatment of non-Hodgkin's B-cell lymphoma. Biochem Soc Trans. 1997;25:705-708; Vose J, Link B, Grossbard M, et al.: Phase II study of rituximab in combination with CHOP chemotherapy in patients with previously untreated intermediate or high-grade non-Hodgkin's lymphoma (NHL). Ann Oncol. 1999;10:58), 30% to 50% of patients with low grade NHL have no clinical response to rituximab(Hainsworth J D, Burris H A, 3rd, Morrissey L H, et al.: Rituximab monoclonal antibody as initial systemic therapy for patients with low-grade non-Hodgkin lymphoma. Blood. 2000;95:3052-3056; Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106. It has been suggested that the level of CD20 expression on lymphoma cells (McLaughlin P, Grillo-Lopez A J, Link B K, et al.: Rituximab chimeric anti-CD20 monoclonal antibody therapy for relapsed indolent lymphoma: half of patients respond to a four-dose treatment program. J Clin Oncol. 1998;16:2825-2833), the presence of high tumor burden at the time of treatment (Coiffier B, Haioun C, Ketterer N, et al.: Rituximab (anti-CD20 monoclonal antibody) for the treatment of patients with relapsing or refractory aggressive lymphoma: a multicenter phase II study. Blood. 1998;92:1927-1932) or low serum rituximab concentrations (McLaughlin P, Grillo-Lopez A J, Link B K, et al.: Rituximab chimeric anti-CD20 monoclonal antibody therapy for relapsed indolent lymphoma: half of patients respond to a four-dose treatment program. J Clin Oncol. 1998;16:2825-2833) may explain the lack of efficacy of rituximab in some patients. Nevertheless, the actual causes of treatment failure remain largely unknown.

The availability of methods allowing the evaluation of patient response to antibody treatment would greatly enhance the therapeutic efficacy of these products. However, the precise mode of action in vivo of such therapeutic antibodies is not clearly documented. Indeed, while in vitro studies suggest various possible modes of action of rituximab (antibody-dependant cell-mediated cytotoxicity (ADCC) (Berinstein N L, Grillo-Lopez A J, White C A, et al.: Association of serum Rituximab (IDEC-C2B8) concentration and anti-tumor response in the treatment of recurrent low-grade or follicular non-Hodgkin's lymphoma. Ann Oncol. 1998;9:995-1001; Harjunpaa A, Junnikkala S, Meri S: Rituximab (anti-CD20) therapy of B-cell lymphomas: direct complement killing is superior to cellular effector mechanisms. Scand J. Immunol. 2000;51:634-641), complement-dependant cytotoxicity (Berinstein N L, Grillo-Lopez A J, White C A, et al.: Association of serum Rituximab (IDEC-C2B8) concentration and anti-tumor response in the treatment of recurrent low-grade or follicular non-Hodgkin's lymphoma. Ann Oncol. 1998;9:995-1001; Reff M E, Carner K, Chambers K S, et al.: Depletion of B cells in vivo by a chimeric mouse human monoclonal antibody to CD20. Blood. 1994; 83:435-445; Idusogie E E, Presta L G, Gazzano-Santoro H, et al.: Mapping of the C1q binding site on rituxan, a chimeric antibody with a human IgG1 Fc. J. Immunol. 2000; 164:4178-4184), direct signalling leading to apoptosis(Golay J, Zaffaroni L, Vaccari T, et al.: Biologic response of B lymphoma cells to anti-CD20 monoclonal antibody rituximab in vitro: CD55 and CD59 regulate complement-mediated cell lysis. Blood. 2000; 95:3900-3908; Shan D, Ledbetter J A, Press OW: Apoptosis of malignant human B cells by ligation of CD20 with monoclonal antibodies. Blood. 1998; 91:1644-1652), etc.; the clear action of these target cell-depleting antibodies in vivo is not documented in humans. Furthermore, while ADCC is an important effector mechanism in the eradication of intracellular pathogens and tumor cells, the role of an ADCC is still controversial(Reff M E, Carner K, Chambers K S, et al.: Depletion of B cells in vivo by a chimeric mouse human monoclonal antibody to CD20. Blood. 1994; 83:435-445; Idusogie E E, Presta L G, Gazzano-Santoro H, et al.: Mapping of the C1q binding site on rituxan, a chimeric antibody with a human IgG1 Fc. J. Immunol. 2000; 164:4178-4184).

The present invention now proposes novel methods and compositions to assess the therapeutic response of a subject to a therapeutic antibody. The invention also proposes methods to select patients having best responding profile to therapeutic antibody treatment. The invention also relates to methods of treating patients with therapeutic antibodies, comprising a prior step of evaluating the patient's response. The invention also relates to compositions and kits suitable to perform the invention. The invention may as well be used in clinical trials or experimental settings, to assess or monitor a subject's response, or to verify the mode of action of an antibody.

The invention is based, in part, on the demonstration of a correlation between the genotype of a subject and its ability to respond to therapeutic antibody treatment. More specifically, the invention shows that the genotype of the FcγRIIIa receptor directly correlates with the subject's response to therapeutic antibody treatment.

Three classes of FcγR (FcγRI, FcγRII and FcγRIII) and their subclasses are encoded by eight genes in humans, all located on the long arm of chromosome 1. Some of these genes display a functional allelic polymorphism generating allotypes with different receptor properties. These polymorphisms have been identified as genetic factors increasing the susceptibility to autoimmune or infectious diseases (Clynes R A, Towers TL, Presta L G, Ravetch J V: Inhibitory Fc receptors modulate in vivo cytoxicity against tumor targets. Nat Med. 2000;6:443-446; Fijen C A, Bredius R G, Kuijper E J, et al.: The role of Fcγ receptor polymorphisms and C3 in the immune defence against Neisseria meningitidis in complement-deficient individuals. Clin Exp Immunol. 2000;120:338-345; Dijstelbloem H M, Scheepers R H, Oost W W, et al.: Fcγ receptor polymorphisms in Wegener's granulomatosis: risk factors for disease relapse. Arthritis Rheum. 1999;42:1823-1827). One of these genetic factors is a gene dimorphism in FCGR3A, which encodes FcγRIIIa with either a phenylalanine (F) or a valine (V) at amino-acid position 158(Myhr K M, Raknes G, Nyland H, Vedeler C: Immunoglobulin G Fc-receptor (FcγR) IIA and IIIB polymorphisms related to disability in MS. Neurology. 1999;52:1771-1776; Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114). This residue directly interacts with the lower hinge region of IgG1 as recently shown by IgG1-FcγRIII co-crystallization(Wu J, Edberg J C, Redecha PB, et al.: A novel polymorphism of FcγRIIIa (CD16) alters receptor function and predisposes to autoimmune disease. J Clin Invest. 1997;100:1059-1070. It has been clearly demonstrated that human IgG1 binds more strongly to homozygous FcγRIIIa-158V natural killer cells MK) than to homozygous FcγRIIIa-158F or heterozygous NK cells(Myhr K M, Raknes G, Nyland H, Vedeler C: Immunoglobulin G Fc-receptor (FcγR) IIA and IIIB polymorphisms related to disability in MS. Neurology. 1999;52:1771-1776.; Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114).

We undertook to evaluate a possible correlation between the FCGR3A genotype and a patient response to therapeutic antibody treatment in vivo. Our invention stems in part from the unexpected discovery that a very strong correlation exists between said genotype and said response profile, the presence of a valine residue at position 158 being indicative of a high response rate. More specifically, the genotyping of FCGR3A was performed in patients with previously untreated follicular NHL who had received rituximab alone, a particular situation in which the response rate is very high (Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106). The FCGR2A-131H/R was also determined as control since this gene co-localizes with FCGR3A on chromosome 1q22 and encodes the macrophage FcγRIIa receptor.

The FCGR3A-158V/F genotype was determined in 47 patients having received rituximab for a previously untreated follicular non-Hodgkin's lymphoma. Clinical and molecular responses were evaluated at two months (N2) and at one year (N12). Positive molecular response was defined as a disappearance of the BCL2-JH gene rearrangement in both peripheral blood and bone marrow. FCGR3A-158V homozygous patients were 21% whereas FCGR3A-158F homozygous and heterozygous patients (FCGR3A-158F carriers) were 34% and 45%, respectively. The objective response rates at M2 and M12 were 100% and 90% in FCGR3A-158V homozygous patients compared with 65% (p=0.02) and 51% (p=0.03) in FCGP3A-158F carriers. A positive molecular response was observed at M12 in 5/6 of homozygous FCGR3A-158V patients compared with 5/16 of FCGR3A-158F carriers (p=0.04). Furthermore, the homozygous FCGR3A-158V genotype was confirmed to be the single parameters associated with clinical and molecular responses in multivariate analysis and was also associated with a lower rate of disease progression (p=0.05).

Accordingly, the present invention establishes, for the first time, an association between the FCGR3A genotype and clinical and molecular responses to therapeutic antibodies. The invention thus provides a first unique marker that can be used to monitor, evaluate or select a patient's response. This invention thus introduces new pharmacogenetical approaches in the management of patients with malignancies, viral infections or other diseases related to the presence of pathological cells in a subject, particularly non-Hodgkin's lymphoma.

An object of this invention resides in a method of assessing the response of a subject to a therapeutic antibody treatment, comprising determining in vitro the FCGR3A genotype and/or the presence of a polymorphism in the FcγRIIIa receptor of said subject. More specifically, the method comprises determining in vitro the FCGR3A158 genotype of said subject.

A further object of this invention is a method of selecting patients for therapeutic antibody treatment, the method comprising determining in vitro the FCGR3A genotype and/or the presence of a polymorphism in the FcγRIIIa receptor of said subject. More specifically, the method comprises determining in vitro the FCGR3A158 genotype of said subject.

Another object of this invention is a method of improving the efficacy or treatment condition or protocol of a therapeutic antibody treatment in a subject, comprising determining in vitro the FCGR3A genotype and/or the presence of a polymorphism in the FcγRIIIa receptor of said subject. More specifically, the method comprises determining in vitro the FCGR3A158 genotype of said subject.

More specifically, determining in vitro the FCGR3A158 genotype of a subject comprises determining amino acid residue at position 158 of FcγRIIIa receptor (or corresponding codon in the FCGR3A gene), a valine at position 158 being indicative of a better response to said treatment and a phenylalanine at position 158 being indicative of a lower response to said treatment.

Within the context of this invention, the term “therapeutic antibody or antibodies” designates more specifically any antibody that functions to deplete target cells in a patient. Specific examples of such target cells include tumor cells, virus-infected cells, allogenic cells, pathological immunocompetent cells (e.g., B lymphocytes, T lymphocytes, antigen-presenting cells, etc.) involved in allergies, autoimmune diseases, allogenic reactions, etc., or even healthy cells (e.g., endothelial cells in an anti-angiogenic therapeutic strategy). Most preferred target cells within the context of this invention are tumor cells and virus-infected cells. The therapeutic antibodies may, for instance, mediate a cytotoxic effect or a cell lysis, particularly by antibody-dependant cell-mediated cytotoxicity (ADCC). ADCC requires leukocyte receptors for the Fc portion of IgG (FcγR) whose function is to link the IgG-sensitized antigens to FcγR-bearing cytotoxic cells and to trigger the cell activation machinery. While this mechanism of action has not been evidenced in vivo in humans, it may account for the efficacy of such target cell-depleting therapeutic antibodies. The therapeutic antibodies may by polyclonal or, preferably, monoclonal. They may be produced by hybridomas or by recombinant cells engineered to express the desired variable and constant domains. The antibodies may by single chain antibodies or other antibody derivatives retaining the antigen specificity and the lower hinge region or a variant thereof. These may be polyfunctional antibodies, recombinant antibodies, ScFv, humanized antibodies, or variants thereof. Therapeutic antibodies are specific for surface antigens, e.g., membrane antigens. Most preferred therapeutic antibodies are specific for tumor antigens (e.g., molecules specifically expressed by tumor cells), such as CD20, CD52, ErbB2 (or HEP2/Neu), CD33, CD22, CD25, MUC-1, CEA, KDR, αVβ3, etc., particularly lymphoma antigens (e.g., CD20). The therapeutic antibodies are preferably IgG1 or IgG3, more preferably IgG1.

Typical examples of therapeutic antibodies of this invention are rituximab, alemtuzumab and trastuzumab. Such antibodies may be used according to clinical protocols that have been authorized for use in human subjects. Additional specific examples of therapeutic antibodies include, for instance, epratuzumab, basillximab, daclizumab, cetuximab, labetuzumab, sevirumab, tuvurimab, palivizumab, infliximab, omalizumab, efalizumab, natalizumab, clenolximab, etc., as listed in Tables 4 or Table 5.

Within the context of the present invention, a subject or patient includes any mammalian subject or patient, more preferably a human subject or patient.

According to the invention the term FCGR3A gene refers to any nucleic acid molecule encoding a FcγRIIIa polypeptide in a subject. This term includes, in particular, genomic DNA, cDNA, RNA (pre-rRNA, messenger RNA, etc.), etc. or any synthetic nucleic acid comprising all or part of the sequence thereof. Synthetic nucleic acid includes cDNA, prepared from RNAs, and containing at least a portion of a sequence of the FCGR3A genomic DNA as for example one or more introns or a portion containing one or more mutations. Most preferably, the term FCGR3A gene refers to genomic DNA, cDNA or mRNA, typically genomic DNA or mRNA. The FCGR3A gene is preferably a human FCγRIIIa gene or nucleic acid, i.e., comprises the sequence of a nucleic acid encoding all or part of a FcγRIIIa polypeptide having the sequence of human FcγRIIIa polypeptide. Such nucleic acids can be isolated or prepared according to known techniques. For instance, they may be isolated from gene libraries or banks, by hybridization techniques. They can also be genetically or chemically synthesized. The genetic organization of a human FCγRIIIa gene is depicted on FIG. 2. The amino acid sequence of human FcγRIIIa is represented FIG. 3. Amino acid position 158 is numbered from residue 1 of the mature protein. It corresponds to residue 176 of the pre-protein having a signal peptide. The sequence of a wild type FCGR3A gene is represented on FIG. 4 (see also Genbank accession Number AL590385 or NM_{—}000569 for partial sequence).

Within the context of this invention, a portion or part means at least 3 nucleotides (e.g., a codon), preferably at least 9 nucleotides, even more preferably at least 15 nucleotides, and can contain as much as 1000 nucleotides. Such a portion can be obtained by any technique well known in the art, e.g., enzymatic and/or chemical cleavage, chemical synthesis or a combination thereof. The sequence of a portion of a FCGR3A gene encoding amino acid position 158 is represented below, for sake of clarity:

| | |
|---|---|
| cDNA | 540 550 560 570 580 |
| genomic DNA | 4970 4980 4990 5000. |
| 158F allele | tcctacttctgcagggggctttttqggagtaaaaatgtgtcttca |
| | S Y F C R G L F G S K N V S S |
| 158V allele | tcctacttctqcagggggcttgttgggagtaaaaatgtgtcttca |
| | S Y F C R G L V G S K N V S S |

As indicated above, the invention comprises a method of determining in vitro the FCGR3A158 genotype of said subject. This more particularly comprises determining the nature of amino acid residue present (or encoded) at position 158 of the FcγRIIIa polypeptide.

Genotyping the FCGR3A gene or corresponding polypeptide in said subject may be achieved by various techniques, comprising analysing the coding nucleic acid molecules or the encoded polypeptide. Analysis may comprise sequencing, migration, electrophoresis, immuno-techniques, amplifications, specific digestions or hybridisations, etc.

In a particular embodiment, determining amino acid residue at position 158 of FcγRIIIa receptor comprises a step of sequencing the FCGR3A receptor gene or RNA or a portion thereof comprising the nucleotides encoding amino acid residue 158.

In another particular embodiment, determining amino acid residue at position 158 of FcγRIIIa receptor comprises a step of amplifying the FCGR3A receptor gene or RNA or a portion thereof comprising the nucleotides encoding amino acid residue 158. Amplification may be performed by polymerase chain reaction (PCR), such as simple PCR, RT-PCR or nested PCR, for instance, using conventional methods and primers.

In this regard, amplification primers for use in this invention more preferably contain less than about 50 nucleotides even more preferably less than 30 nucleotides, typically less than about 25 or 20 nucleotides. Also, preferred primers usually contain at least 5, preferably at least 8 nucleotides, to ensure specificity. The sequence of the primer can be prepared based on the sequence of the FCGR3A gene, to allow full complementarity therewith, preferably. The probe may be labeled using any known techniques such as radioactivity, fluorescence, enzymatic, chemical, etc. This labeling can use for example Phosphor 32, biotin (16-dUTP), digoxygenin (11-dUTP). It should be understood that the present invention shall not be bound or limited by particular detection or labeling techniques. The primers may further comprise restriction sites to introduce allele-specific restriction sites in the amplified nucleic acids, as disclosed below.

Specific examples of such amplification primers are, for instance, SEQ ID NO: 1-4.

It should be understood that other primers can be designed by the skilled artisan, such as any fragment of the FCGR3A gene, for use in the amplification step and especially a pair of primers comprising a forward sequence and a reverse sequence wherein said primers of said pair hybridize with a region of a FCGRA gene and allow amplification of at least a portion of the FCGR3A gene containing codon 158. In a preferred embodiment, each pair of primers comprises at least one primer that is complementary, and overlaps with codon 158, and allows to discriminate between 158V (gtt) and 158F (ttt). The amplification conditions may also be adjusted by the skilled person, based on common general knowledge and the guidance contained in the specification.

In a particular embodiment, the method of the present invention thus comprises a PCR amplification of a portion of the FCGR3a mRNA or gDNA with specific oligonucleotide primers, in the cell or in the biological sample, said portion comprising codon 158, and a direct or indirect analysis of PCR products, e.g., by electrophoresis, particularly Denaturing Gel Gradient Electrophoresis (DGGE).

In another particular embodiment, determining amino acid residue at position 158 of FcγRIIIa receptor comprises a step of allele-specific restriction enzyme digestion. This can be done by using restriction enzymes that cleave the coding sequence of a particular allele (e.g., the 158V allele) and that do not cleave the other allele (e.g., the 158F allele, or vice versa). Where such allele-specific restriction enzyme sites are not present naturally in the sequence, they may be introduced therein artificially, by amplifying the nucleic acid with allele-specific amplification primers containing such a site in their sequence. Upon amplification, determining the presence of an allele may be carried out by analyzing the digestion products, for instance by electrophoresis. This technique also allows to discriminate subjects that are homozygous or heterozygous for the selected allele.

Examples of allele-specific amplification primers include for instance SEQ ID NO: 3. SEQ ID NO:3 introduces the first 3 nucleotides of the NlaIII site (5′-CATG-3′). Cleavage occurs after G. This primer comprises 11 bases that do not hybridise with FCGR3A, that extend the primer in order to facilitate electrophoretic analysis of the amplification products) and 21 bases that hybridise to FCGR3A, except for nucleotide 31 (A) which creates the restriction site.

In a further particular embodiment, determining amino acid residue at position 158 of FcγRIIIa receptor comprises a step of hybridization of the FCGR3A receptor gene or RNA or a portion thereof comprising the nucleotides encoding amino acid residue 158, with a nucleic acid probe specific for the genotype Valine or Phenylalanine, and determining the presence or absence of hybrids.

It should be understood that the above methods can be used either alone or in various combinations. Furthermore, other techniques known to the skilled person may be used as well to determine the FCGR3A158 genotype, such as any method employing amplification (e.g. PCR), specific primers, specific probes, migration, etc., typically quantitative RT-PCR, LCR (Ligase Chain Reaction), TMA (Transcription Mediated Amplification), PCE (an enzyme amplified immunoassay) and bDNA (branched DNA signal amplification) assays.

In a preferred embodiment of this invention, determining amino acid residue at position 158 of FcγRIIIa receptor comprises:
Obtaining genomic DNA from a biological sample,
Amplifying the FcγRIIIa receptor gene or a portion thereof comprising the nucleotides encoding amino acid residue 158, and
determining amino acid residue at position 158 of said FcγRIIIa receptor gene.

Amplification can be accomplished with any specific technique such as PCR, including nested PCR, using specific primers as described above. In a most preferred embodiment, determining amino acid residue at position 158 is performed by allele-specific restriction enzyme digestion. In that case, the method comprises:
Obtaining genomic DNA from a biological sample,
Amplifying the FcγRIIIa receptor gene or a portion thereof comprising the nucleotides encoding amino acid residue 158,
Introducing an allele-specific restriction site,
Digesting the nucleic acids with the enzyme specific for said restriction site and,
Analysing the digestion products, i.e., by electrophoresis, the presence of digestion products being indicative of the presence of the allele.

In another particular embodiment, the genotype is determined by a method comprising: total (or messenger) RNA extraction from cell or biological sample or biological fluid in vitro or ex vivo, optionally cDNA synthesis, (PCR) amplification with FCGR3A-specific oligonucleotide primers, and analysis of PCR products.

The method of this invention may also comprise determining amino acid residue at position 158 of FcγRIIIa receptor directly by sequencing the FcγRIIIa receptor polypeptide or a portion thereof comprising amino acid residue 158 or by using reagents specific for each allele of the FcγRIIIa polypeptide. This can be determined by any suitable technique known to the skilled artisan, including by immuno-assay (ELISA, EIA, RIA, etc.). This can be made using any affinity reagent specific for an FcγRIIIa158 polypeptide, more preferably any antibody or fragment or derivative thereof. In a particular embodiment, the FcγRIIIa158 polypeptide is detected with an anti-FcγRIIIa158 antibody (or a fragment thereof) that discriminates between FcγRIIIa158V and FcγRIIIa158F, more preferably a monoclonal antibody. The antibody (or affinity reagent) may be labelled by any suitable method (radioactivity, fluorescence, enzymatic, chemical, etc.). Alternatively, FcγRIIIa158 antibody immune complexes may be revealed (and/or quantified) using a second reagent (e.g., antibody), labelled, that binds to the anti-FcγRIIIa158 antibody, for instance.

The above methods are based on the genotyping of FCGR3A158 in a biological sample of the subject. The biological sample may be any sample containing a FCGR3A gene or corresponding polypeptide, particularly blood, bone marrow, lymph node or a fluid, particularly blood or urine, that contains a FCGR3A158 gene or polypeptide. Furthermore, because the FCGR3A158 gene is generally present within the cells, tissues or fluids mentioned above, the method of this invention usually uses a sample treated to render the gene or polypeptide available for detection or analysis. Treatment may comprise any conventional fixation techniques, cell lysis (mechanical or chemical or physical), or any other conventional method used in immunohistology or biology, for instance.

The method is particularly suited to determine the response of a subject to an anti-tumor therapeutic antibody treatment. In this regard, in a particular embodiment, the subject has a tumor and the therapeutic antibody treatment aims at reducing the tumor burden, particularly at depleting the tumor cells. More preferably, the tumor is a lymphoma, such as more preferably a B lymphoma, particularly a NHL. As indicated above, the antibody is preferably an IgG1 or an IgG3, particularly an anti-CD20 IgG1 or IgG3, further preferably a humanized antibody, for instance rituximab.

The invention also relates to a bispecific antibody, wherein said bispecific antibody specifically binds CD16 and a tumor antigen, for instance a CD20 antigen. The invention also encompasses pharmaceutical compositions comprising such a bispecific antibody and a pharmaceutically acceptable excipient or adjuvant.

The subject invention also provides for modified therapeutic antibodies (“altered antibodies”) that comprise modifications preferably, in the Fc region that modify the binding affinity of the antibody to one or more FcγR. Methods for modifying antibodies with modified binding to one or more FcγR are known in the art, see, e.g., PCT Publication Nos. WO 2004/016750 (International Application PCT/US2003/025399), WO 99/158572, WO 99/151642, WO 98/123289, WO 89/107142, WO 88/107089, and U.S. Pat. Nos. 5,843,597 and 5,642,821, each of which is incorporated herein by reference in their entirety.

Therapeutic antibodies identified in Table 4, such as CDP571 (Ceiltech, plc (Berkshire, UK); used to treat Crohn's disease and rheumatoid arthritis), D2E7 (Cambridge Antibody Technology Group, plc (Cambridge, UK)/BASF (Ludwigshafen, Germany)); used to treat rheumatoid arthritis), or Infliximab (Centocor, Inc., Malvern, P A; used to treat Crohn's disease and rheumatoid arthritis), or those antibodies disclosed in Tables 6A, 6B and 7 of International Patent Application PCT/US2003/025399 (which is hereby incorporated by reference in its entirety and is reproduced as Tables 5-7 of the subject application) can be modified as taught in the above and below identified applications and used for the treatment of diseases for which such antibodies are typically used. In some embodiments, the invention provides altered antibodies that have altered affinity, either higher or lower affinity, for an activating FcγR, e.g., FcγRIII. In certain preferred embodiments, altered antibodies having higher affinity for FcγR are provided. Preferably such modifications also have an altered Fc-mediated effector function.

Modifications that affect Fc-mediated effector function are well known in the art (See U.S. Pat. No. 6,194,351, which is incorporated herein by reference in its entirety). The amino acids that can be modified include but are not limited to proline 329, proline 331, and lysine 322. Proline 329 and/or 331 and lysine 322 can, preferably be replaced with alanine, however, substitution with any other amino acid is also contemplated. See International Publication No.: WO 00/142072 and U.S. Pat. No. 6,194,551 which are incorporated herein by reference in their entirety.

Thus, modification of the Fc region can comprise one or more alterations to the amino acids found in the antibody Fc region. Such alterations can result in an antibody with an altered antibody-mediated effector function, an altered binding to other Fc receptors (e.g., Fc activation receptors), an altered ADCC activity, an altered Clq binding activity, an altered complement dependent cytotoxicity activity, or any combination thereof.

The invention also provides antibodies with altered oligosaccharide content. As used herein, oligosaccharides refer to carbohydrates containing two or more simple 20 sugars and the two terms may be used interchangeably herein. Carbohydrate moieties of the instant invention will be described with reference to commonly used nomenclature in the art. For a review of carbohydrate chemistry, see, e.g., Hubbard et al., 198 1 Ann. Rev. Biochem., 50: 555-583, which is incorporated herein by reference in its entirety. This nomenclature includes for example, Man which represents mannose; GlcNAc which represents 2-N-acetylglucosamine; Gal which represents galactose; Fuc for fucose and Glc for glucose. Sialic acids are described by the shorthand notation NeuNAc for 5-N-acetylneuraminic acid, and NeuNGc for 5-glycolneuraminic.

Antibodies typically contain carbohydrate moieties at conserved positions in the constant region of the heavy chain, and up to 30% of human IgGs have a glycosylated Fab region. IgG has a single N-linked biantennary carbohydrate structure at Asn 297 which resides in the CH2 domain (Jefferis et al., 1998, Immunol. Rev. 163: 59-76; Wright et al., 1997, Trends Biotech 15: 26-32). Human IgG typically has a carbohydrate of the following structure; GlcNAc(Fucose)-GlcNAc-Man-(ManGlcNAc)₂. However variations among IgGs in carbohydrate content do occur which leads to altered function, see, e.g., Jassal et al, 35 2001 Biochem. Biophys. Res. Commun. 288: 243-9; Groenink et al., 1996 J. Immunol. 26: 37 1404-7; Boyd et al., 1995 Mol. Immunol. 32: 13 1 1-8; Kumpel et al., 1994, Human Antibody Hybridomas, 5: 143-5 1.

Antibodies comprising a variation in the carbohydrate moiety that is attached to Asn 297 are also provided by the subject invention. The carbohydrate moiety can have a galactose and/or galactose-sialic acid at one or both of the terminal GlcNAc and/or a third GlcNac chain. In some embodiments, the antibodies are substantially free of one or more selected sugar groups, e.g., one or more sialic acid residues, one or more galactose residues, and/or one or more fucose residues. An antibody that is substantially free of one or more selected sugar groups may be prepared using common methods known to one skilled in the art, including for example recombinantly producing an antibody of the invention in a host cell that is defective in the addition of the selected sugar groups(s) to the carbohydrate moiety of the antibody, such that about 90-100% of the antibody in the composition lacks the selected sugar group(s) attached to the carbohydrate moiety.

Alternative methods for preparing such antibodies include for example, culturing cells under conditions which prevent or reduce the addition of one or more selected sugar groups, or post-translational removal of one or more selected sugar groups. In a specific embodiment, a method of producing a substantially homogenous antibody preparation, wherein about 80-100% of the antibody in the composition lacks a fucose on its carbohydrate moiety, e.g., the carbohydrate attachment on Asn 297 is provided. The antibody may be prepared for example by (a) use of an engineered host cell that is deficient in fucose metabolism such that it has a reduced ability to fucosylate proteins expressed therein; (b) culturing cells under conditions which prevent or reduce fusocylation; (c) post-translational removal of fucose, e.g., with a fucosidase enzyme; or (d) purification of the antibody so as to select for the product which is not fucosylated. Most preferably, nucleic acid encoding the desired antibody is expressed in a host cell that has a reduced ability to fucosylate the antibody expressed therein.

Host cells for the production of such antibodies are, preferably, dihydrofolate reductase (DHR) deficient. DHR deficient Chinese hamster ovary cells (CHO) are known in the art, e.g., a Lec 13 CHO cell (Oectin resistant CHO mutant cell line; Ribka & Stanley, 1986, Somatic Cell & Molec. Gen. 12(1): 51-62; Ripka et al., 1986 Arch. Biochem. Biophys. 249(2): 533-45), CHO-K1, DUX-B11, CHO-DP12 or CHO-DG44. Such cells can be modified so that the antibody is not substantially fucosylated. Thus, the cell may display altered expression and/or activity for the fucoysltransferase enzyme, or another enzyme or substrate involved in adding fucose to the N-linked oligosaccharide so that the enzyme has a diminished activity and/or reduced expression level in the cell. For methods to produce antibodies with altered fucose content, see, e.g., WO 03/035835 and Shields et al., 2002, J. Biol. Chem. 277(30): 26733-40; both of which are incorporated herein by reference in their entirety.

The altered carbohydrate modifications can modulate one or more of the following characteristics of the antibody: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (see Shields R. L. et al., 2001, 10 J. Biol. Chem. 277(30): 26733-40; Davies J. et al., 2001, Biotechnology & Bioengineering, 74(4): 288-294).

The altered carbohydrate modifications enhance the binding of antibodies of the invention to FcγR receptors (e.g., FcγRIIIA). Altering carbohydrate modifications in accordance with the methods of the invention includes, for example, increasing the carbohydrate content of the antibody or decreasing the carbohydrate content of the antibody. Methods of altering carbohydrate contents are known to those skilled in the art, see, e.g., Wallick et al., 1988, Journal of Exp. Med. 168(3): 1099-1 109; Tao et al., 1989 Journal of Immunology, 143(8): 2595-2601; Routledge et al., 1995 Transplantation, 60(8): 847-53; Elliott et al. 2003; Nature Biotechnology, 2 1: 4 14-2 1; Shields et al. 2002 Journal of Biological Chemistry, 277(30): 26733-40; all of which are incorporated herein by reference in their entirety. In some aspects of the invention, antibodies comprising one or more glycosylation sites, so that one or more carbohydrate moieties are covalently attached to the antibody are provided. In other embodiments, the invention encompasses antibodies comprising one or more glycosylation sites and one or more modifications in the Fc region, such as those disclosed supra and those known to one skilled in the art. In preferred embodiments, the one or more modifications in the Fc region enhance the affinity of the antibody for an activating FcγR, e.g., FcγIIIA, relative to the antibody comprising the wild type Fc regions. Antibodies of the invention with one or more glycosylation sites and/or one or more modifications in the Fc region have an enhanced antibody mediated effector function, e.g., enhanced ADCC activity or NK activating activity.

In some embodiments, the invention further comprises antibodies comprising one or more modifications of amino acids that are directly or indirectly known to interact with a carbohydrate moiety of the antibody, including but not limited to amino acids at positions 241, 243, 244, 245, 245, 249, 256, 258, 260, 262, 264, 265, 296, 299, and 301. Amino acids that directly or indirectly interact with a carbohydrate moiety of an antibody are known in the art, see, e.g., Jefferis et al., 1995 Immunology Letters, 44: 11 1-7, which is incorporated herein by reference in its entirety.

Antibodies that have been modified by introducing one or more glycosylation sites into one or more sites of the antibodies, preferably without altering the functionality of the antibody, e.g., binding activity to FcγRIII can also be used in the practice of the subject invention. Glycosylation sites may be introduced into the variable and/or constant region of the antibodies of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N-or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. The antibodies of the invention may comprise one or more glycosylation sites, including N-linked and O-linked glycosylation sites. Any glycosylation site for N-linked or O-linked glycosylation known in the art may be used in accordance with the instant invention. An exemplary N-linked glycosylation site that is useful in accordance with the methods of the present invention, is the amino acid sequence: Asn-X-Thr/Ser, wherein X may be any amino acid and Thr/Ser indicates a threonine or a serine. Such a site or sites may be introduced into an antibody of the invention using methods well known in the art to which this invention pertains. See, for example, “In Vitro Mutagenesis,” Recombinant DNA: A Short Course, J. D. Watson, et al. W.H. Freeman and Company, New York, 1983, chapter 8, pp. 106-116, which is incorporated herein by reference in its entirety.

An exemplary method for introducing a glycosylation site into an antibody of the invention may comprise: modifying or mutating an amino acid sequence of the antibody so that the desired Asn-X-Thr/Ser sequence is obtained. In some embodiments, the invention encompasses methods of modifying the carbohydrate content of an antibody of the invention by adding or deleting a glycosylation site. Methods for modifying the carbohydrate content of antibodies are well known in the art and encompassed within the invention, see, e.g., U.S. Pat. No. 6,218,149; EP 0 359 096 B1; U.S. Publication No. U.S. 200210028486; WO 03/035835; U.S. Publication No. 2003/0115614; U.S. Pat. No. 6,218,149; U.S. Pat. No. 6,472,511; all of which are incorporated herein by reference in their entirety. In other embodiments, the invention encompasses methods of modifying the carbohydrate content of an antibody of the invention by deleting one or more endogenous carbohydrate moieties of the antibody.

The invention further encompasses methods of modifying an effector function of an antibody of the invention, wherein the method comprises modifying the carbohydrate content of the antibody using the methods disclosed herein or known in the art. Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues. The present invention also encompasses antibodies or fragments thereof comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of the anti-TNFα antibodies discussed herein. In another preferred embodiment, the invention provides “altered antibodies” or fragments thereof that specifically bind FcγR (e.g., FcγR found on NK cells) with greater affinity than unaltered antibodies or fragments thereof that specifically bind to the same FcγR.

The subject application also provides methods of increasing the ADCC activity of NK cells comprising the administration of a composition comprising altered therapeutic antibodies that exhibit increased binding to FcγRIII. Methods of treating diseases, such as those indicated in Tables 4-7 of the subject application, are also provided. In this aspect of the invention, therapeutically effective compositions comprising altered therapeutic antibodies are administered to subjects in need of treatment for diseases such as those disclosed in Tables 4-7 of the subject application. In various embodiments of this aspect of the invention, it is possible to administer lower dosages or amounts of the altered therapeutic antibodies, as compared to the same unaltered therapeutic antibodies, since the binding affinities of the altered therapeutic antibodies are higher.

Thus, as provided herein, the subject application provides the following:
A) A method of improving the efficacy or treatment condition or protocol of Crohn's disease comprising the steps of: a) determining the FCGR3A158 genotype of a subject; b) determining C-reactive protein (CRP) levels in a subject; and c) administering an anti-TNFα antibody to said subject if the subject is exhibits elevated levels of CRP and is homozygous for valine at position 158 of the FcγRIIIa receptor;
B) A method according to A), wherein said anti-TNFα antibody is infliximab;
C) A method of any one of A) or B), wherein determining amino acid residue at position 158 of FcγRIIIa receptor comprises a step of sequencing the FcγRIIIa receptor gene or RNA or a portion thereof comprising the nucleotides encoding amino acid residue 158;
D) A method of any one of A), B) or C), wherein determining amino acid residue at position 158 of FcγRIIIa receptor comprises a step of amplifying the FcγRIIIa receptor gene or RNA or a portion thereof comprising the nucleotides encoding amino acid residue 158;
E) A method of any one of A), B), C) or D), wherein amplification is performed by polymerase chain reaction (PCR), such as PCR, RT-PCR and nested PCR;
F) A method of any one of A), B), C), D) or E), wherein determining amino acid residue at position 158 of FcγRIIIa receptor comprises a step of allele-specific restriction enzyme digestion;
G) A method of any one of A), B), C), D), E), or F), wherein determining amino acid residue at position 158 of FcγRIIIa receptor comprises a step of hybridization of the FcγRIIIa receptor gene or RNA or a portion thereof comprising the nucleotides encoding amino acid residue 158, with a nucleic acid probe specific for the genotype Valine or Phenylalanine;
H) A method of any one of A), B), C), D), E), F) or G), wherein determining amino acid residue at position 158 of FcγRIIIa receptor comprises:
   Obtaining genomic DNA from a biological sample,
   Amplifying the FcγRIIa receptor gene or a portion thereof comprising the nucleotides encoding amino acid residue 158, and
   determining amino acid residue at position 158 of said FcγRIIIa receptor gene;
I) A method of any one of A), B), C), D), E), F), G) or H), wherein determining amino acid residue at position 158 of FcγRIIIa receptor comprises:
   Obtaining genomic DNA from a biological sample,
   Amplifying the FcγRIIIa receptor gene or a portion thereof comprising the nucleotides encoding amino acid residue 158,
   Introducing an allele-specific restriction site,
   Digesting the nucleic acids with the enzyme specific for said restriction site and,
   Analysing the digestion products, i.e., by electrophoresis, the presence of digestion products being indicative of the presence of the allele;
J) A method of any one of A), B), C), D), E), F), G), H) or I), wherein determining amino acid residue at position 158 of FcγRIIIa receptor comprises: total (or messenger) RNA extraction from cell or biological sample or biological fluid in vitro or ex vivo, optionally cDNA synthesis, (PCR) amplification with specific FCGRIIIa oligonucleotide primers, and analysis of PCR products;
K) A method of any one of claims A) or B), wherein determining amino acid residue at position 158 of FcγRIIIa receptor comprises a step of sequencing the FcγRIIIa receptor polypeptide or a portion thereof comprising amino acid residue 158;
L) A method of any one of A), B), C), D), E), F), G), H), I), J) or K) wherein the subject is a human subject;
M) A method of any one A), B), C), D), E), F), G), H), I), J), K) or L), wherein the antibody is an IgG1 or an IgG3;
N) A method according to any one of A), B), C), D), E), F), G), H), I), J), K) or L), wherein CRP levels are determined by an assay that measures the level of CRP or an assay that measures the level of nucleic acid that encodes CRP;
O) A method according to N), wherein said assay measures the level of CRP and is an immunoassay;
P) A method according to N), wherein said assay measures the level of nucleic acid encoding CRP.

Further aspects and advantages of this invention will be disclosed in the following examples, which should be regarded as illustrative and not limiting the scope of this application.

### BRIEF DESCRIPTION OF DRAWINGS

FIG. 1: Adjusted KAPLAN-MEIER estimates of progression-free survival after rituximab treatment according to FcγR3a-158V/F genotype (p=0.05).

FIG. 2: Genetic organization of the human FCGR3A gene.

FIG. 3: Amino acid sequences of human FcγRIIIa158F (SEQ ID NO:7).

FIG. 4: Nucleic acid sequence of human FCGR3A158F (SEQ ID NO:8).

FIG. 5: Biological response to infliximab in patients with elevated C-reactive protein (CRP) (greater than twice the upper limit of the normal range) before treatment according to FCGR3A-158 genotype. Complete and partial biological responses were defined as a normalization of CRP or a decrease of at least 25%, respectively, 4 weeks after treatment. CI, confidence interval; RR, relative risk.

FIGS. 6A and 6B: Variation in the relative value of C-reactive protein (CRP) 4 weeks after infliximab treatment (compared with baseline) according to FCGR3A-158 genotype. (A) Relative CRP variation in F carriers (V/F and F/F genotypes); the median variation was −63.2% (range, +1100% to −98.1%). (B) Relative CRP variation in V/V patients; the median variation was −80.1% (range, −31.0% to −94.8%).

FIG. 7: Clinical response to infliximab according to FCGR3A-158 genotype in a sub-group of patients with elevated C-reactive protein (CRP) (greater than twice the upper limit of the normal range) before treatment. For luminal Crohn's disease (n=113), complete and partial responders were defined by a Crohn's disease activity index (CDAI) below 150 or a decrease of at least 70 points 4 weeks after treatment, respectively. For fistulizing Crohn's disease (n=32), patients were considered as complete or partial responders at week 10 according to complete fistula closure or a decrease of at least 50% in fistula drainage at two consecutive visits, respectively.

### MATERIALS AND METHODS

Patients and treatment

Clinical trial design, eligibility criteria and end-point assessment have been previously reported. (Colombat P, Salles G, Brousse N, et al.: Rituximab (anti-CD20 monoclonal antibody) as first-line therapy of follicular lymphoma patients with low tumor burden: clinical and molecular evaluation. Blood. 2001; 97:101-106). In brief, patients were eligible for inclusion in this study if they had previously untreated follicular CD20 positive NHL according to the REAL classification(Harris N L, Jaffe E S, Stein H, et al.: A revised European-American classification of lymphoid neoplasms: a proposal from the International Lymphoma Study Group. Blood. 1994; 84:1361-1392). Patients were required to present with stage II to IV disease according to Ann-Arbor classification and at least one measurable disease site. All patients were required to have low tumor burden according to the GELF criteria (Brice P, Bastion Y, Lepage E, et at: Comparison in low-tumor-burden follicular lymphomas between an initial no-treatment policy, prednimustine, or interferon alfa: a randomized study from the Groupe d'Etude des Lymphomes Folliculaires. Groupe d'Etude des Lymphomes de l'Adulte. J Clin Oncol. 1997;15:1110-1117). A total of four 375 mg/m² doses of rituximab (Roche, Neuilly, France) were administered by intravenous infusion (days 1, 8, 15, 22). The management of infusion and adverse events has already been reported (Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106). The study protocol was approved by an ethics committee, and all patients gave their informed consent.

Monitoring and Endpoints

Baseline evaluation included clinical examination, chest X-ray, computed tomography (CT) of the chest, abdomen and pelvis, and unilateral bone marrow biopsy. Response was assessed by an independent panel of radiologists who reviewed all the CT scans of the included patients.

The primary efficacy endpoint was the objective response rate, i.e., the proportion of patients achieving either complete remission (CR), unconfirmed CR(CRu) or partial response (PR) according to the criteria recently proposed by an international expert committee (Cheson B D, Horning S J, Coiffier B, et al.: Report of an international workshop to standardize response criteria for non-Hodgkin's lymphomas. NCI Sponsored International Working. J Clin Oncol. 1999;17:1244). Clinical response was evaluated at days 50 and 78. Only the maximum response was taken into account and that assessment time point named M2. All patients were evaluated for progression at one year (M12). Patients in CR or CRu with disappearance of bone marrow infiltration at M2 and reappearance of lymphoma cells in bone marrow at M12 were considered “progressive”; patients in PR with negative bone marrow biopsy at M2 and positive biopsy at M12 were considered in PR.

Molecular analysis of BCL2-JH gene rearrangement was performed by PCR, as previously described, (Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106) on a lymph node obtained at diagnosis and on both peripheral blood and bone marrow at diagnosis, M2 and M12.

FCGR3A-158V/F Genotyping

Out of the 50 patients included in the clinical trial, one patient was excluded after histological review and DNA was not available for two other patients. Forty seven patients were therefore available for FCGR3A genotype analysis. All samples were analysed in the same laboratory and DNA was extracted using standard procedures including precautions to avoid cross-contamination. DNA was isolated from peripheral blood (n=43), bone marrow (n=3) or lymph node (n=1). Genotyping of FCGR3A-158V/F polymorphism was performed as described by Koene et al. ((Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114) with a nested PCR followed by an allele-specific restriction enzyme digestion. Briefly, two FCGR3A specific primers (5′-ATATITACAGAATGGCACAGG-3′, SEQ ID NO: 1; 5′-GACTTGGTACCCAGGTTGAA-3′, SEQ ID NO: 2) (Eurobio, Les Ulis, France) were used to amplify a 1.2 kb fragment containing the polymorphic site. The PCR assay was performed with 1.25 μg of genomic DNA, 200 ng of each primer, 200 μmol/L of each dNTP (MBI Fermentas, Vilnius, Lithuania) and 1 U of Taq DNA polymerase (Promega, Charbonnière, France) as recommended by the manufacturer. This first PCR consisted in 10 min at 95° C., then 35 cycles (each consisting in 3 steps at 95° C. for 1 min, 57° C. for 1.5 min, 72° C. for 1.5 min) and 8 min at 72° C. to achieve complete extension. The second PCR used primers (5′-ATCAGATTCGATCCTACTTCTGCAGGGGGCAT-3′ SEQ ID NO: 3; 5′-ACGTGCTGAGCTTGAGTGATGGTGATGTTCAC-3′ SEQ ID NO: 4) (Eurobio) amplifying a 94 bp fragment and creating a NlaIII restriction site only in the FCGR3A-158V allele. This nested PCR was performed with 1 μL of the amplified DNA, 150 ng of each primer, 200 μmol/L of each dNTP and 1 U of Taq DNA polymerase. The first cycle consisted in 5 min at 95° C. then 35 cycles (each consisting in 3 steps at 95° C. for 1 min, 64° C. for 1 min, 72° C. for 1 min) and 9.5 min at 72° C. to complete extension. The amplified DNA (10 μL) was then digested with 10 U of NlaIII (New England Biolabs, Hitchin, England) for 12 h at 37° C. and separated by electrophoresis on a 8% polyacrylamide gel. After staining with ethidium bromide, DNA bands were visualized with UV light. For homozygous FCGR3A-158F patients, only one undigested band (94 bp) was visible. Three bands (94 bp, 61 bp and 33 bp) were seen in heterozygous individuals whereas for homozygous FCGR3A-158V patients, only two digested bands (61 bp and 33 bp) were obtained.

FCGR2-131H/R Genotyping

Genotyping of FCGR2A-131H/R was done by PCR followed by an allele-specific restriction enzyme digestion according to Liang et al. X M, Arepally G, Poncz M, McKenzie S E: Rapid detection of the Fc γ RIIA-H/R 131 ligand-binding polymorphism using an allele-specific restriction enzyme digestion (ASRED). J Immunol Methods. 1996;199:55-59). The sense primer (5′-GGAAAATCCCAGAAATTCTCGC-3′ SEQ ID NO: 5) (Eurobio) has been modified to create a BstUI restriction site in case of R allele whereas the antisense primer (5′-CAACAGCCTGACTACCTATTACGCGGG-3′ SEQ ID NO: 6) Eurobio) has been modified to carry a second BstUI restriction site that served as an internal control. PCR amplification was performed in a 50 μL reaction with 1.25 μg genomic DNA, 170 ng of each primer, 200 μmol/L of each dNTP, 0.5 U of Taq DNA polymerase, and the manufacturer's buffer. The first cycle consisted of 3 minutes at 94° C. followed by 35 cycles (each consisting in 3 steps at 94° C. for 15 seconds, 55° C. for 30 seconds, 72° C. for 40 seconds) and 7 min at 72° C. to complete extension. The amplified DNA (7 μL) was then digested with 20 U of BstUI (New England Biolabs) for 12 h at 60° C. Further analysis was performed as described for FCGR3A genotyping. The FCGR2A-131H and -131R alleles were visualized as a 337 bp and 316 bp DNA fragments, respectively.

Statistical Analysis

Clinical and biological characteristics as well as clinical and molecular responses of the patients in the different genotypic groups were compared using a Chi-squared test or by Fisher's exact test when appropriated. A logistic regression analysis including: sex, age (> or ≦60 years), number of extra-nodal sites involved (≧ or <2), bone marrow involvement, BCL2-JH rearrangement status at diagnosis and FCGR3A genotype was used to identify independent prognostic variables influencing clinical and molecular responses. Progression-free survival was calculated according to the method of Kaplan and Meier (Kaplan E, Meier P: Nonparametric estimation from incomplete observations. J Am Stat Assoc. 1958;53:457-481) and was measured from the start of treatment until progression/relapse or death. Comparison of the progression-free survival by FCGR3A genotype was performed using the log-rank test. P<0.05 was considered as statistically significant.

### Results

Clinical Response

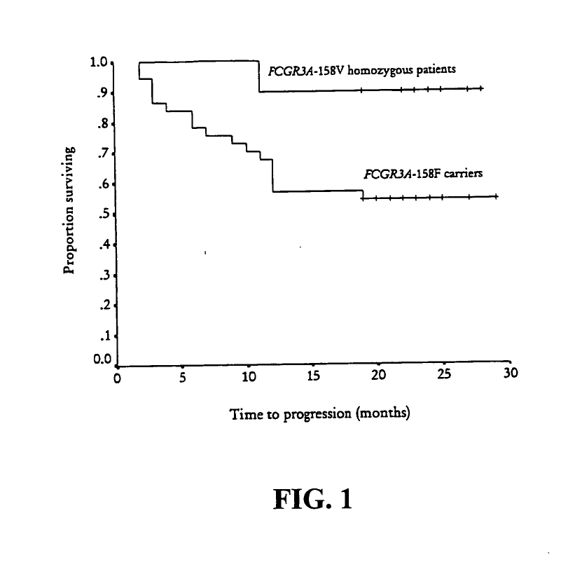

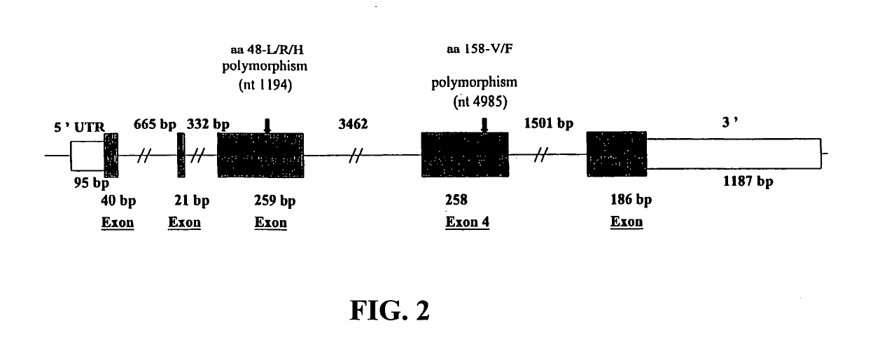

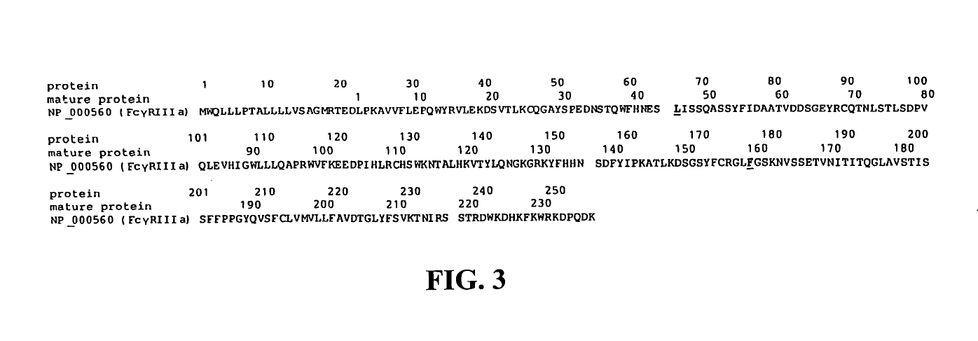

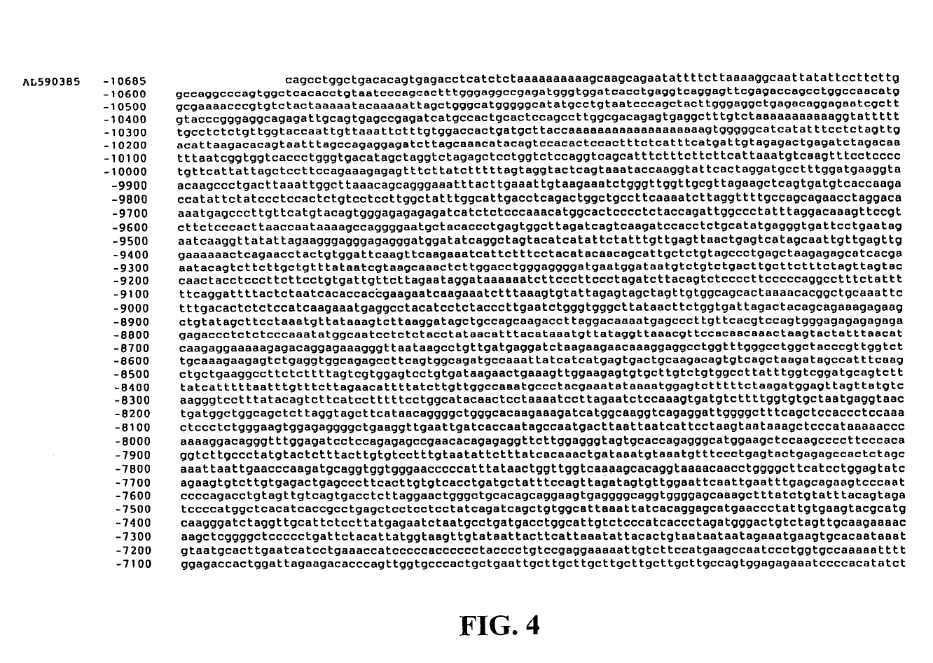

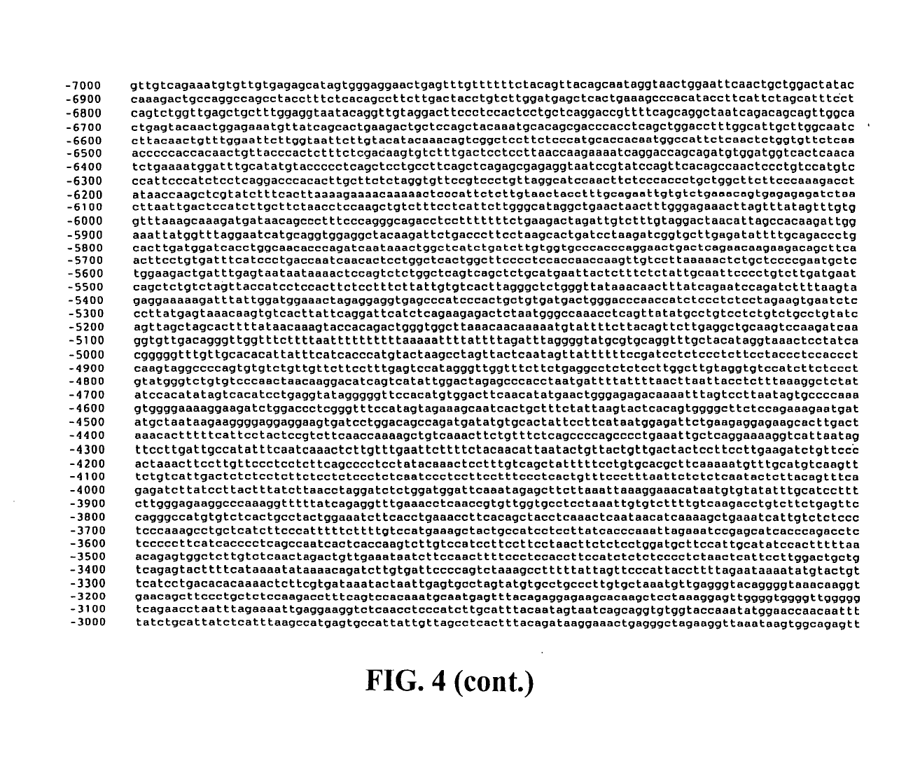

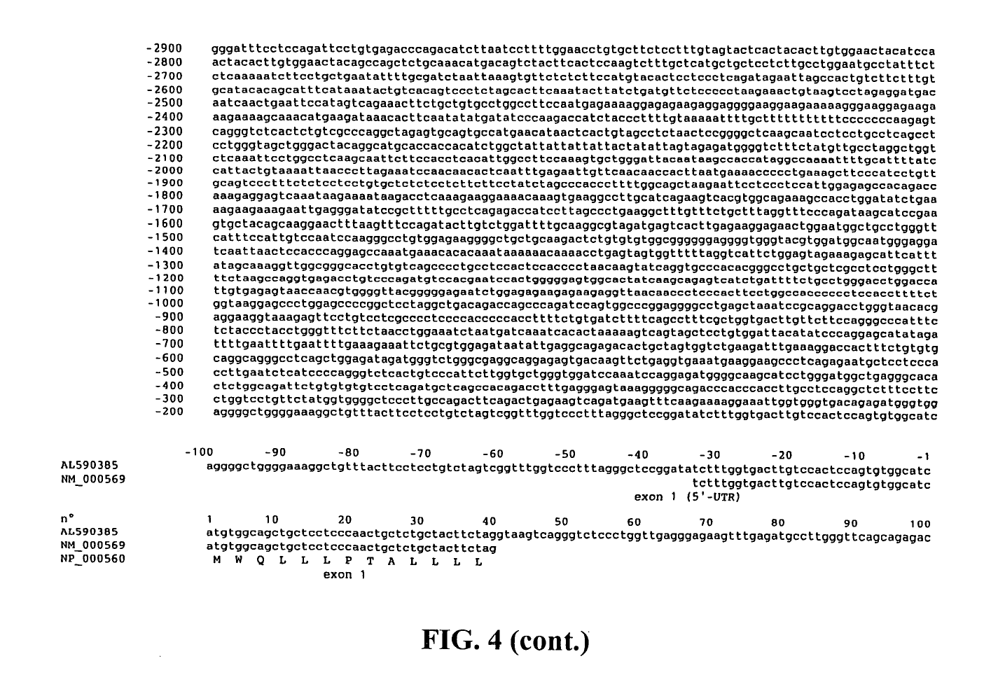

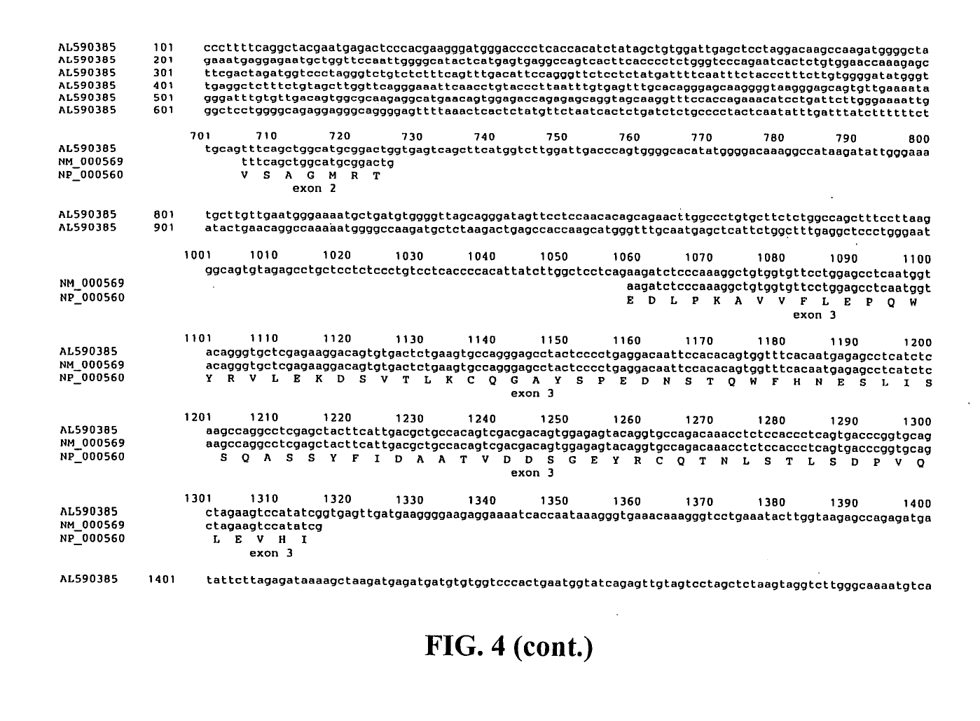

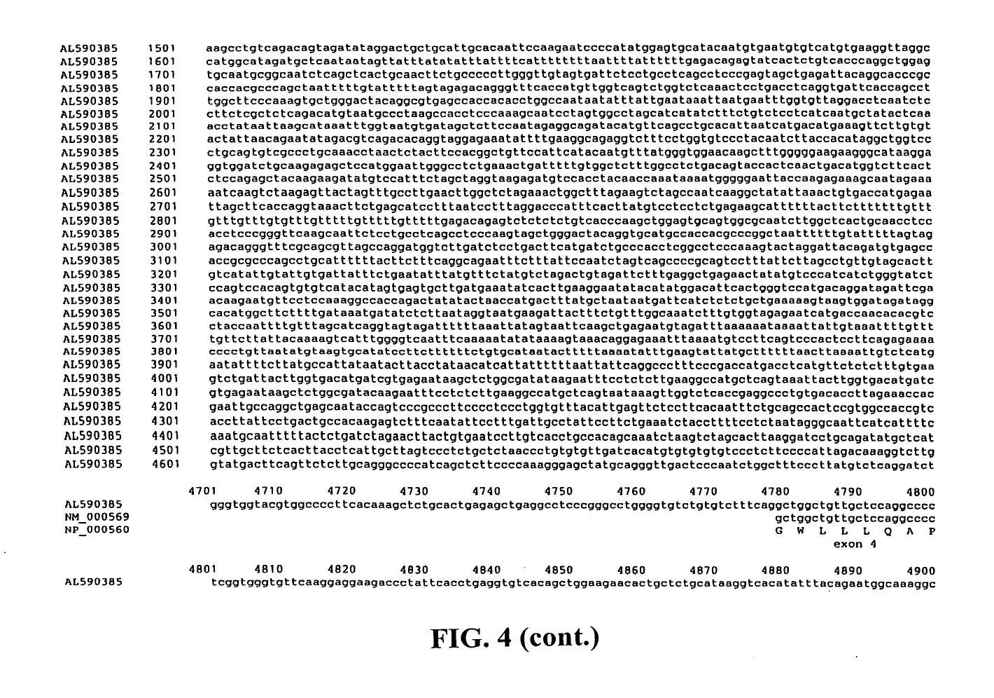

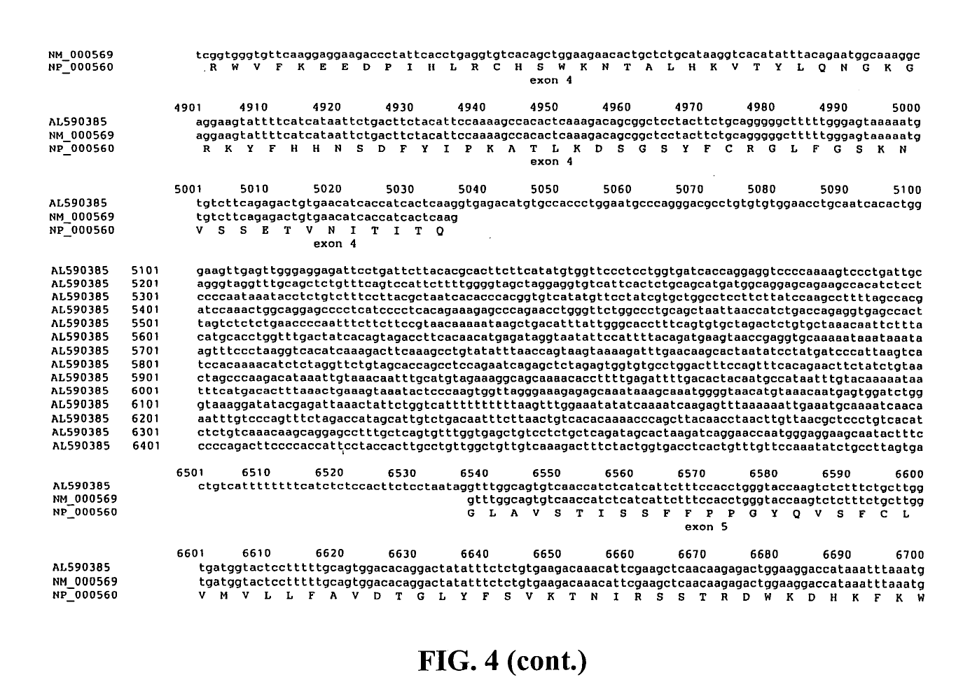

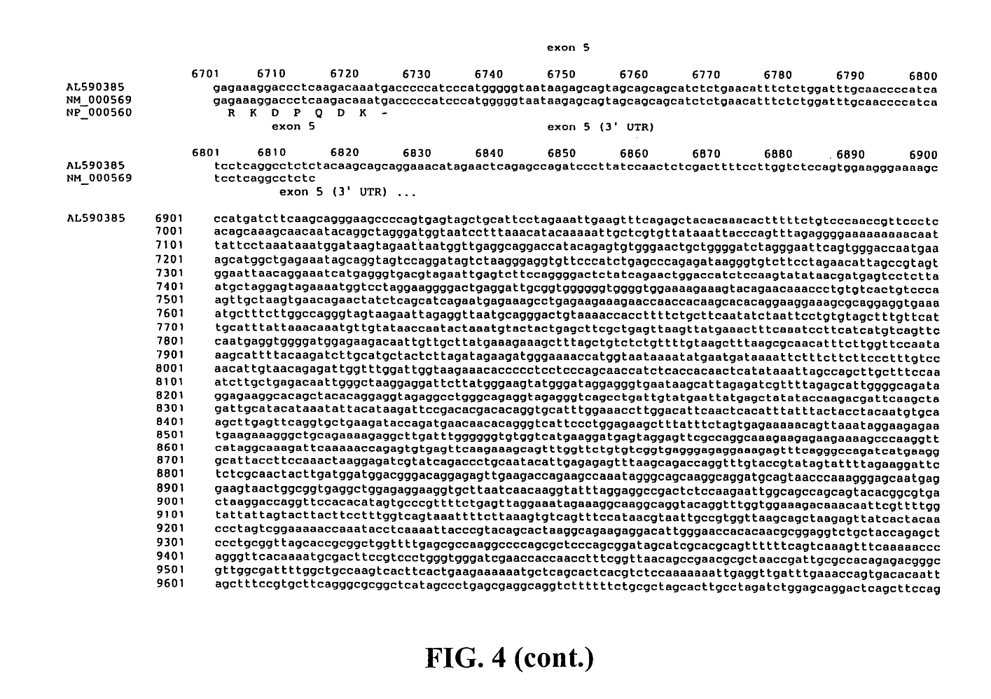

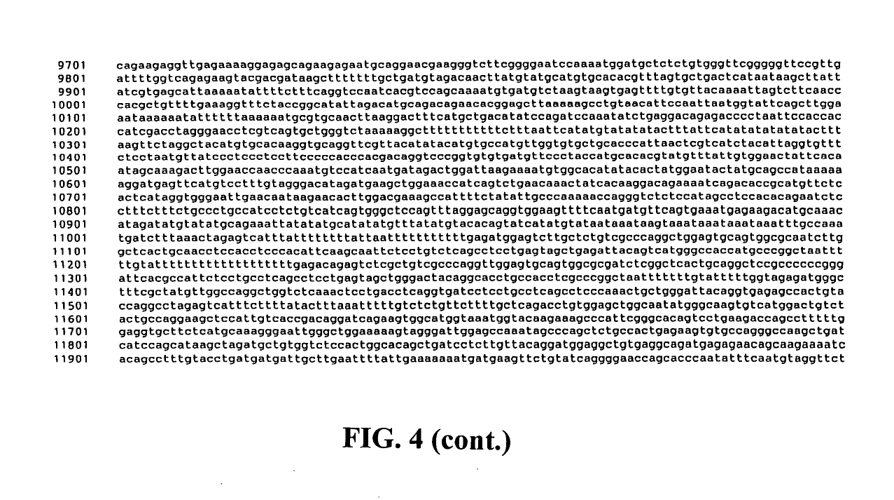

Out of the 49 patients tested for the FCGR3A-158V/F polymorphism, 10 (20%) and 17 (35%) were homozygous for FCGR3A-158V and FCGR3A-158F, respectively, and 22 (45%) were heterozygous. The three groups were not different in terms of sex, disease stage, bone marrow involvement, number of extra-nodal sites involved or presence of BC2-JH rearrangement in peripheral blood and bone marrow at diagnosis (Table 1). No difference was found when homozygous FCGR3A-158V patients were compared with FCGR3A-158F carriers (FCGR3A-158F homozygous and heterozygous patients) or when homozygous FCGR3A-158F patients were compared with FCGR3A-158V carriers (FCGR3A-158V homozygous and heterozygous patients). The objective response rate at M2 was 100% (CR+CRu=40%), 70% (CR+CRu=29%) and 64% (CR+CRu=18%) in FCGR3A-158V homozygous, FCGR3A-158F homozygous and heterozygous patients respectively (P=0.09). A significant difference in objective response rate was observed between FCGR3A-158V homozygous patients and FCGR3A-158F carriers with 67% (CR+CRu=23%) objective response rate for this latter group (relative risk=1.5; 95% CI, 1.2-1.9; P-0.03) (Table 2). No difference was observed between FCGR3A-158F homozygous patients and FCGR3A-158V carriers. At M12, the objective response rate was 90% (CR+CRu=70%), 59% (CR+CRu=35%) and 45% (CR+CRu=32%) in FCGR3A-158V homozygous, FCGR3A-158F homozygous and heterozygous patients respectively (P=0.06). The difference in objective response rate was still present one year after treatment between FCGR3A-158V homozygous group and FCGR3A-158F carriers with 51% (CR+CRu=33%) objective response rate for this latter group (relative risk=1.7; 95% CI, 1.2-2.5; P=0.03). The logistic regression analysis showed that the homozygous FCGR3A-158V genotype was the only predictive factor for clinical response both at M2 (P0.02) and at M12 (P=0.01). The progression-free survival at 3 years (median follow-up: 35 months; 31-41) (FIG. 1) was 56% in FCGR3A-158V homozygous patients and 35% in FCGR3A-158F carriers (ns). Out of the 45 patients analyzed for FCGR2A-131H/R polymorphism, 9 (20%) and 13 (29%) were homozygous for FCGR2A-131R and FCGR2A-131H, respectively, while 23 (51%) were heterozygous. There was no difference in the characteristics at inclusion or clinical response to rituximab treatment for these three groups or for homozygous FCGR2A-131H patients and FCGR2A-131R carriers, or for homozygous FCGR2A-131R patients and FCGR2A-131H carriers (data not shown).

Molecular Response

At diagnosis, BCL2-JH rearrangement was detected in both peripheral blood and in bone marrow in 30 (64%) patients, enabling further follow-up. Twenty-five patients (six FCGR3A-158V homozygous patients and 19 FCGR3A-158F carriers) and 23 patients (six FCGR3A-158V homozygous patients and 17 FCGR3A-158F carriers) were analysed for BCL2-JH rearrangement in both peripheral blood and bone marrow at M2 and at M12 (fable 3). At M2, a cleaning of BCL2-JH rearrangement was observed in 3/6 of the FCGR3A-158V homozygous patients and in 5/19 of the FCGR3A-158F carriers (ns). In contrast, the rate of BC2-JH rearrangement cleaning at M12 was higher (5/6) in the FCGR3A-158V homozygous patients than in the FCGR3A-158F carriers (5/17) (relative risk=2.8; 95% CI, 1.2-6.4; P=0.03). The logistic regression analysis showed that the FCGR3A-158V homozygous genotype was the only factor associated with a greater probability of exhibiting BCL2-JH rearrangement cleaning at M12 (P=0.04). The single homozygous FCGR3A-158V patient still presenting with BCL2-JH rearrangement in peripheral blood and bone marrow at M12 was in CR 23 months after rituximab treatment. In contrast, the molecular responses at M2 and M12 were not influenced by the FCGR2A-131H/R polymorphism (data not shown).

### Discussion

Because of the increasing use of rituximab in B cell lymphoproliferative malignancies, enhanced understanding of treatment failures and of the mode of action of rituximab is required. In this regard, we genotyped FCGR3A in follicular NHL patients with well-defined clinical and laboratory characteristics and treated with rituximab alone (Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106). In particular, all the patients included in this study had a low tumor burden NHL and a molecular analysis of BCL2-JH at diagnosis and during follow-up. The FCGR3A allele frequencies in this population were similar to those of a general Caucasian population (Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114; Wu J, Edberg J C, Redecha P B, et al.: A novel polymorphism of FcγRIIIa (CD16) alters receptor function and predisposes to autoimmune disease. J Clin Invest. 1997;100:1059-1070). Our results show an association between the FCGR3A genotype and the response to rituximab. Indeed, homozygous FCGR3A-158V patients, who account for one fifth of the population, had a greater probability of experiencing clinical response, with 100% and 90% objective response rates at M2 and M12, respectively. Moreover, five of the six FCGR3A-158V homozygous patients analysed for BCL2-JH rearrangement showed molecular response at M12, compared to 5 of the 17 FCGR3A-158F carriers. FCGR3A-158V homozygosity was the only factor associated with the clinical and molecular responses. However, these higher clinical and molecular responses were still unsufficient to significantly improve the progression-free survival in homozygous FCGR3A-158V patients.

This is the first report of an easily assessable genetic predictive factor for both clinical and molecular responses to rituximab. However, the genetic association does not demonstrate the mode of action of rituximab involves FcγRIIIa. The association observed between FCGR3A genotype and response to rituximab might be due to another genetic polymorphism in linkage disequilibrium. Those polymorphisms could be located in FCGR3A itself like the triallelic FCGR3A-48L/H/R polymorphism (de Haas M, Koene H R, Kleijer M, et al.: A triallelic Fc γ receptor type IIIA polymorphism influences the binding of human IgG by NK cell Fc γ RIIIa. J. Immunol. 1996;156:3948-3955) or in other FcγR-coding genes, since FCGR3A is located on the long arm of chromosome 1, which includes the three FCGR2 genes and FCGR3B (Peltz G A, Grundy H O, Lebo R V, Yssel H, Barsh G S, Moore K W: Human Fc γ RIII: cloning, expression, and identification of the chromosomal locus of two Fc receptors for IgG. Proc Natl Acad Sci USA. 1989;86:1013-1017). A linkage disequilibrium has been reported between FCGR2A and FCGR3B (Schnackenberg L, Flesch B K, Neppert J: Linkage disequilibria between Duffy blood groups, Fc γ IIa and Fc γ IIIb allotypes. Exp Clin Immunogenet. 1997;14:235-242). However, the fact that FCGR2A-131H/R polymorphism was not associated with a better response to rituximab strongly supports the fact that a gene very close to FCGR3A or FCGR3A itself is directly involved.

Several in vitro studies argue in favor of direct involvement of FCGR3A-158V/F polymorphism. First, Koene et al (Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114) have shown that the previously reported differences in IgG binding among the three FcγRIIIa-48L/H/R isoforms (de Haas M, Koene H R, Kleijer M, et al.: A triallelic Fc γ receptor type IIIA polymorphism influences the binding of human IgG by NK cell Fc γ RIIa. J. Immunol. 1996;156:3948-3955) are a consequence of the linked FcγRIIIa-158V/F polymorphism and several teams have demonstrated that NK cells from individuals homozygous for the FCGR3A-158V allotype have a higher affinity for human complexed IgG1 and are more cytotoxic towards IgG1-sensitized targets (Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114; Wu J, Edberg J C, Redecha P B, et al.: A novel polymorphism of FcγRIIIa (CD16) alters receptor function and predisposes to autoimmune disease. J Clin Invest. 1997;100:1059-1070; Sondemmann P, Huber R, Oosthuizen V, Jacob U: The 3.2-A crystal structure of the human IgG1 Fc fragment-Fc γRIII complex. Nature. 2000;406:267-273). Our present results establish that FCGR3A-158V homozygous patients have a better response to rituximab, which is probably due to a better in vivo binding of that chimeric human IgG1 to FcγRIIIa. Secondly, NK cell- and macrophage-mediated ADCC is one of the mechanisms triggered by anti-CD20 antibodies in vitro (Anderson D R, Grillo-Lopez A, Varns C, Chambers K S, Hanna N: Targeted anti-cancer therapy using rituximab, a chimaeric anti-CD20 antibody (IDEC-C2B8) in the treatment of non-Hodgkin's B-cell lymphoma. Biochem Soc Trans. 1997;25:705-708; Harjunpaa A, Junnikkala S, Meri S: Rituximab (anti-CD20) therapy of B-cell lymphomas: direct complement killing is superior to cellular effector mechanisms. Scand J. Immunol. 2000;51:634-641; Reff M E, Carner K, Chambers K S, et al: Depletion of B cells in vivo by a chimeric mouse human monoclonal antibody to CD20. Blood. 1994;83:435-445) as well as in murine models in vivo (Hooijberg E, Sein J J, van den Berk P C, et al.: Eradication of large human B cell tumors in nude mice with unconjugated CD20 monoclonal antibodies and interleukin 2. Cancer Res. 1995;55:2627-2634; Funakoshi S, Longo D L, Murphy W J: Differential in vitro and in vitro antitumor effects mediated by anti-CD40 and anti-CD20 monoclonal antibodies against human B-cell lymphomas. J Immunother Emphasis Tumor Immunol. 1996;19:93-101; Clynes R A, Towers T L, Presta L G, Ravetch J V: Inhibitory Fc receptors modulate in vivo cytoxicity against tumor targets. Nat Med. 2000;6:443-446), and rituximab-mediated apoptosis is amplified by FcγR-expressing cells (Shan D, Ledbetter J A, Press OW: Apoptosis of malignant human B cells by ligation of CD20 with monoclonal antibodies. Blood. 1998;91:1644-1652; Shan D, Ledbetter J A, Press OW: Signaling events involved in anti-CD20-induced apoptosis of malignant human B cells. Cancer Immunol Immunother. 2000;48:673-683). Out of all FcγR, FcγRIIIa is the only receptor shared by NK cells and macrophages. We thus postulate that FCGR3A-158V patients show a better response to rituximab because they have better ADCC activity against lymphoma cells. The fact that more than 50% of the FCGR3A-158F carriers nonetheless present a clinical response to rituximab could be explained by lower, but still sufficient, ADCC activity or, more likely, by other mechanisms operating in vivo such as complement-dependent cytotoxicity, complement-dependent cell-mediated cytotoxicity (Harjunpaa A, Junnikkala S, Meri S: Rituximab (anti-CD20) therapy of B-cell lymphomas: direct complement killing is superior to cellular effector mechanisms. Scand J. Immunol. 2000;51:634-641; Idusogie E E, Presta L G, Gazzano-Santoro H, et al.: Mapping of the C1q binding site on rituxan, a chimeric antibody with a human IgG1 Fc. J. Immunol. 2000;164:4178-4184; Golay J, Zaffaroni L, Vaccari T, et al.: Biologic response of B lymphoma cells to anti-CD20 monoclonal antibody rituximab in vitro: CD55 and CD59 regulate complement-mediated cell lysis. Blood. 2000;95:3900-3908) and/or apoptosis (Shan D, Ledbetter J A, Press OW: Apoptosis of malignant human B cells by ligation of CD20 with monoclonal antibodies. Blood. 1998;91:1644-1652; Shan D, Ledbetter J A, Press OW: Signaling events involved in anti-CD20-induced apoptosis of malignant human B cells. Cancer Immunol Immunother. 2000;48:673-683). ADCC could then be viewed as an additional mechanism in the response to rituximab that is particularly effective in FCGR3A-158V homozygous patients.

The in vitro studies suggest a “gene-dose” effect with a level of IgG1 binding to NK cells from FCGR3A heterozygous donors intermediate between that observed with NK cells from FCGR3A-158V and FCGR3A-158F homozygotes (Koene HR, Kleijer M, Algra J, Roos D, von dem Borne A E, de Haas M: Fc γRIIIa-158V/F polymorphism influences the binding of IgG by natural killer cell Fc γRIIa, independently of the Fc γRIIIa-48L/R/H phenotype. Blood. 1997;90:1109-1114). However, the clinical response of heterozygous patients appears similar to that of FCGR3A-158F homozygous patients. Further studies with larger groups of patients will be required to conclude against a “gene-dose” effect in vivo.

Since FcγRIIIa is strongly associated with a better response to rituximab, it needs to be taken into account in the development of new drugs targeting the CD20 antigen. For example, it may be possible to use engineered rituximab to treat FCGR3A-158F-carrier patients with B cell lymphomas. Indeed, by modifying various residues in the IgG1 lower hinge region, Shields et al have recently obtained IgG1 mutants which bind more strongly to FcγRIIIa-158F than native IgG1 (Shields R L, Namenuk A K, Hong K, et al.: High resolution mapping of the binding site on human IgG1 for Fc y RI, Fc γ RII, Fc γ RIII, and FcRn and design of IgG1 variants with improved binding to the Fc γ R. J Biol. Chem. 2001;276:6591-6604).

Taken together, these results allow settingup new therapeutic strategies against B lymphoproliferative disorders based upon prior determination of the patients FCGR3A genotype. Since this polymorphism has the same distribution in various ethnic population, including blacks and Japanese, such a strategy may be applied worldwide (Koene H R, Kleijer M, Algra J, Roos D, von dem Borne A E, de Haas M: Fc γRIIIa-158V/F polymorphism influences the binding of IgG by natural killer cell Fc γRIIIa, independently of the Fc γRIIIa-48L/R/H phenotype. Blood. 1997;90:1109-1114; Leppers-van de Straat F G, van der Pol W, Jansen M D, et al.: A novel PCR-based method for direct Fcγ receptor IIIa (CD16) allotyping. J Immunol Methods. 2000;242:127-132; Lehrnbecher T, Foster C B, Zhu S, et al.: Variant genotypes of the low-affinity Fcγ receptors in two control populations and a review of low-affinity Fcγ receptor polymorphisms in control and disease populations. Blood. 1999;94:4220-4232). Furthermore, such a pharmacogenetic approach may also be applied to other intact humanized IgG1 antibodies used in the treatment of B cell malignancies, such as Campath-1H, or those used in the treatment of other malignancies, such as trastuzumab (Herceptin®). Even more generally, this approach may apply to other intact (humanized) therapeutic (IgG1) antibodies developed to deplete target cells.

### Example 2

Infliximab is a chimeric monoclonal immunoglobulin G1 (IgG1) antibody against tumour necrosis factor-α (TNF-α) (Knight D M, Trinh H, Le J, et al. Construction and initial characterization of a human-mouse chimeric antibody. Mol Immunol 1993; 30: 1443-53). It is effective in refractory and fistulizing Crohn's disease (Targan S, Hanauer S, van Deventer S J H, et al. A short term study of chimeric antibody cA2 to tumor necrosis factor alpha for Crohn's disease. N Engl J Med 1997; 337: 1029-35; Present DH, Rutgeerts P, Targan S, et a. Infliximab for the treatment of fistulas in patients with Crohn's disease. N Eng J Med 1999; 340: 1398-405). In controlled trials, as well as in routine practice, the response rate to first treatment is around 75%, with half the responders showing a complete response and half a partial clinical response (Targan S, Hanauer S, van Deventer S J H, et al. A short term study of chimeric antibody cA2 to tumor necrosis factor alpha for Crohn's disease. N Engl J Med 1997; 337: 1029-35; Present D H, Rutgeerts P, Targan S, et al. Infliximab for the treatment of fistulas in patients with Crohn's disease. N Engl J Med 1999; 340: 1398-405; Cohen R D, Tsang J F, Hanauer S B. Infliximab in Crohn's disease: first anniversary clinical experience. Am J Gastroenterol 2000; 95: 3469-77; Farrell R J, Shah S A, Lodhavia P J, et al. Clinical experience with infliximab therapy in 100 patients with Crohn's disease. Am J Gastroenterol 2000; 95: 3490-7). Certain demographic and clinical characteristics, including a young age (Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63), colonic location of the disease (Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63), co-treatment with immunosuppressive drugs (Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63; Parsi M A, Achkar J P, Richardson S, et al. Predictors of response to infliximab in patients with Crohn's disease. Gastroenterology 2002; 123: 707-13). and non-smoking, (Parsi M A, Achkar J P, Richardson S, et al. Predictors of response to infliximab in patients with Crohn's disease. Gastroenterology 2002; 123: 707-13) have been associated with a positive response to the drug. Furthermore, some patients seem to be biologically predisposed to a non-response to infliximab, as manifested by the inefficacy of re-treatment in these patients (Targan S, Hanauer S, van Deventer S J H, et al. A short term study of chimeric antibody cA2 to tumor necrosis factor alpha for Crohn's disease. N Engl J Med 1997; 337: 1029-35; Taylor K D, Plevy S E, Yang H, et al Anca pattern and LTA haplotype relationship to clinical responses to anti-TNF antibody treatment in Crohn's disease. Gastroenterology 2001; 120: 1347-55) We have now associated the FCGF3A-158 polymorphism to biological and clinical responses to infliximab in Crohn's disease.

Methods

Study population

Two hundred patients with Crohn's disease, included between November 1998 and August 2000 in an expanded access programme of infliximab in Belgium, were studied. All patients were treated with infliximab for the first time. To be included in the expanded access programme, patients had to be between 18 and 65 years of age, adopt adequate birth control measures, give informed consent and have one of the following specific inclusion criteria: (i) a single or multiple perianal or enterocutaneous draining fistula(e) as a complication of Crohn's disease, resistant to conventional treatment for at least 3 months; (ii) moderate to severely active Crohn's disease of at least 6 months' duration, with colitis, ileitis or ileo-colitis confirmed by radiography or endoscopy, refractory or dependent on oral steroid therapy (>8 mg/day prednisone equivalent) and/or not responding to immunosuppressive agents (azathioprine, 6-mercaptopurine or methotrexate). Approval from the Ethics Committee was obtained in April 1998 and patients gave written informed consent for both infliximab treatment and pharmacogenetic ancillary studies. The demographic and clinical characteristics of the patients are given in Table 8. There were two sub-groups of patients: 142 patients with active luminal (non-fistulizing) disease and 58 patients with fistulizing disease. Luminal Crohn's disease patients were treated with a single infusion of infliximab (5 mg/kg). These patients were followed prospectively for 12 weeks, with physical examination, Crohn's disease activity index (CDAI) calculation and C-reactive protein (CRP) measurement (by routine procedure) at weeks 0, 4, 8 and 12. Patients with fistulizing disease were treated with three consecutive infusions (5 mg/kg) at weeks 0, 2 and 6. These patients were followed prospectively for 18 weeks, with physical examination (including fistulous track drainage), CDAI calculation and CRP measurement (by routine procedure) at weeks 0, 2, 6, 10, 14 and 18.

Classification of Response to Infliximab

The response to infliximab was assessed on the basis of both clinical and biological evolution. Clinically, patients were classified as complete responders, partial responders or non-responders according to published controlled clinical trials (Targan S, Hanauer S, van Deventer S J H, et al. A short term study of chimeric antibody cA2 to tumor necrosis factor alpha for Crohn's disease. N Engl J Med 1997; 337: 1029-35; Present D H, Rutgeerts P, Targan S, et al. Infliximab for the treatment of fistulas in patients with Crohn's disease. N En J Med 1999; 340: 1398-405). In non-fistulizing disease, patients were considered to be complete or partial responders at week 4 according to a decrease in CDAI below 150 or a decrease of 70 points from baseline, respectively. Furthermore, according to a recent National Institutes of Health Consensus Conference, we also looked at a partial response defined as a decrease of at least 100 points from baseline. In fistulizing disease, patients were considered to be complete or partial responders at week 10 according to complete fistula closure or a decrease of at least 50% in fistula drainage at two consecutive visits, respectively. Furthermore, the variation in CDAI between pre-treatment (week 0) and post-treatment (week 4 or 10) periods was calculated for all patients.

Biologically, patients were classified as complete, partial or non-responders at 4 weeks (non-fistulizing disease) or 10 weeks (fistulizing disease) on the basis of CRP evolution (measured by a routine procedure using immunoturbidimetry in local laboratories). This analysis was performed on a sub-group of 145 patients for whom CRP values were available before and after treatment and who showed an elevated CRP (greater than twice the upper limit of the normal range) before treatment. Patients were classified as complete or partial responders according to the normalization of CRP after treatment or a decrease of at least 25% from the baseline level, respectively. Furthermore, for all patients, the variation of CRP (absolute and relative values) was calculated between pre-treatment (week 0) and post-treatment (week 4 or 10) periods.

FCGR3A-158V/F Genotyping

Genomic DNA was isolated using phenol/chloroform extraction, as described previously (Vermeire S, Louis E, Rutgeerts P, et al. NOD2/CARD15 does not influence response to infliximab in Crohn's disease. Gastroenterology 2002; 123: 106-11), and stored at −80° C. until use. Allele-specific polymerase chain reactions (PCRs) were performed with primers partially based on those previously designed by Leppers-van de Straat et al. (Leppers-van de Straat F G, van der Pol W L, Jansen M D, et al. A novel PCR-based method for direct Fc gamma receptor IIIa (CD16) allotyping. J Immunol Methods 2000; 242: 127-32) and recently described (Dall'Ozzo S, Andrès C, Bardos P, Watier H, Thibault G. Rapid single-step FCGR3A genotyping based on SYBR Green I fluorescence in real-time multiplex allele-specific PCR. J Immunol Methods 2003; 277: 185-92). The forward primer (5′-TCCAAAAGCCACACTCAAAGTC-3′) includes a 3′ penultimate mismatch (underlined) to completely avoid FCGR3B amplification and to allow FCGR3A-specific amplification. The V allele-specific (5′-GGGGGGCCCCGGGGGTGATGTTCACAGTCTGAGAAGACACAJTITFACTCCCTAC-3) and the F allele-specific (5′-AGACACATTTTTACTCCCATA-3′) (Eurobio, Les Ulis, France) reverse primers also included new 3′ mismatches (underlined) to enhance allele specificity. Moreover, the V allele reverse primer was 5′ elongated (italic characters) to allow single-tube amplification and allele discrimination after electrophoresis. PCRs were carried out in a final volume of 25 μL consisting of Taq polymerase buffer (670 mM Tris-HCl pH 8.8, 160 nm (NH)₂SO₄, 0.1% Tween 20), 2 ml MgCl₂, 400 μM DNTP (MBI Fermentas, Vilnius, Lithuania), 20 pmol of the common forward primer, 14.5 pmol of the V allele-specific reverse primer and 30 pmol of the F allele-specific reverse primer (all three synthesized by Genset S A, Paris, France), 1 unit of Taq DNA polymerase (Eurobio) and 2 μL genomic DNA. PCR was set up in a GeneAmp PCR system 2400 (Perkin Elmer France SA, Saint Quentin en Yvelines, France) programmed for an initial denaturation step of 5 min at 95° C., followed by 35 cycles at 94° C. for 20 s, hybridization at 58° C. for 20 s and elongation at 72° C. for 20 s. PCR products were then analysed by electrophoresis in 8% polyacrylamide gels run in TBE buffer (90 mM) Tris-HCl, 90 mM boric acid, 2.5 mM ethylenediaminetetra-acetic acid) (Eurobio). After staining with ethidium bromide (Eurobio), gels were visualized using ultraviolet transillumination (Gel Doc 1000 system, Bio-Rad, Hercules, Calif., USA) and images were captured on a Kodak Digital Science Image (Kodak, Rochester, N.Y., USA). The F and V allele amplification products appeared as bands of 70 bp and 102 bp, respectively.

Statistical Analyses

CRP values were normalized and expressed as a multiple of the upper limit of the normal range determined in each laboratory (95% reference interval, as recommended by the International Federation of Clinical Chemistry(Solberg H E. Approved recommendation on the theory of reference values. Part 5. Statistical treatment of collected reference values. Determination of reference limits. J Clin Chem C/in Biochem 1987; 25: 645-56)). Response rates according to various FCGR3A-158V/F genotypes were compared using the chi-squared test. Comparisons were performed between the three genotypes (V/V, V/F, F/F) and also between V carriers (V/V, V/F) and non-carriers (F/F), as well as between F carriers (F/F, V/F) and non-carriers (V/V). Variations in CDAI between pre- and post-treatment periods (δCDAI) and variations in CRP between pre- and post-treatment periods (δCRP) were compared by t-test or Mann-Whitney test as required.

Multivariate analyses by stepwise logistic regression were performed. An initial analysis was performed, with the clinical response as the dependent variable, on a sub-group of 189 patients for whom all the demographic, clinical and biological data were available. A second analysis was performed, with the biological response as the dependent variable, on a sub-group of 145 patients who showed an elevated CRP before treatment. A third multivariate analysis, with the clinical response as the dependent variable, was also performed on this sub-group of 145 patients. P values of <0.05 were considered to be significant.

Results

Clinical and Biological Responses to Infliximab

Clinically, there were 106 (53.0%), 43 (21.5%) and 51 (25.5%) complete, partial and non-responders, respectively, in the study. In patients with non-fistulizing disease, there were 79 (55.6%), 30 (21.1%) and 33 (23.3%) complete, partial and non-responders, respectively. In patients with fistulizing disease, there were 27 (46.6%), 13 (22.4%) and 18 (31.0%) complete, partial and non-responders, respectively. Biologically, as determined by the decrease in serum CRP, there were 52 (35.9%), 58 (40.0%) and 35 (24.1%) complete, partial and non-responders, respectively.

Clinical and Biological Responses to Infliximab According to FCGR3A-158V/F Genotypes

There were 35 (17.5%), 100 (50.0%) and 65 (32.5%) FCGR3A-158V/V, V/F and F/F patients, respectively. The frequencies of clinical complete, partial and non-responders, according to the FCGR3A genotype, are shown in Table 9. There was no significant difference when comparing the various genotypes or V and F carriers and non-carriers. Even when looking at partial responses defined using more stringent criteria (decrease of at least 100 points in CDAI), there was still no significant difference between the groups (80.0% responders in V/V patients vs. 63.0% responders in V/F and F/F patients; P=0.11). The median 6CDAI was −161 (range, +65 to −427) in V/V homozygous patients vs. −116 (range, +135 to −349) in the F carrier group (P=0.5). The median duration of response was 14 weeks (range, 6-104 weeks) in the V/V genotype vs. 12 weeks (range, 1-192 weeks) in the V/F and F/F genotypes (P=0.9).

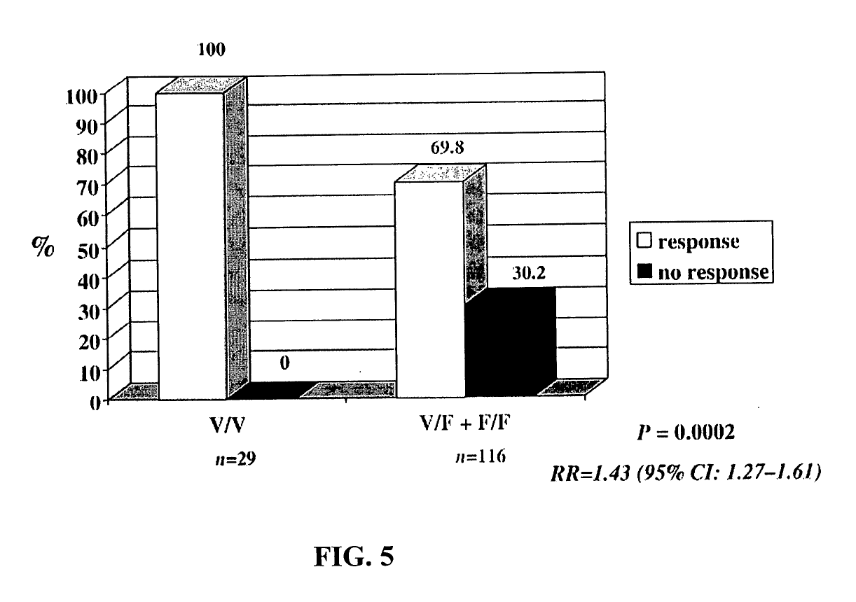

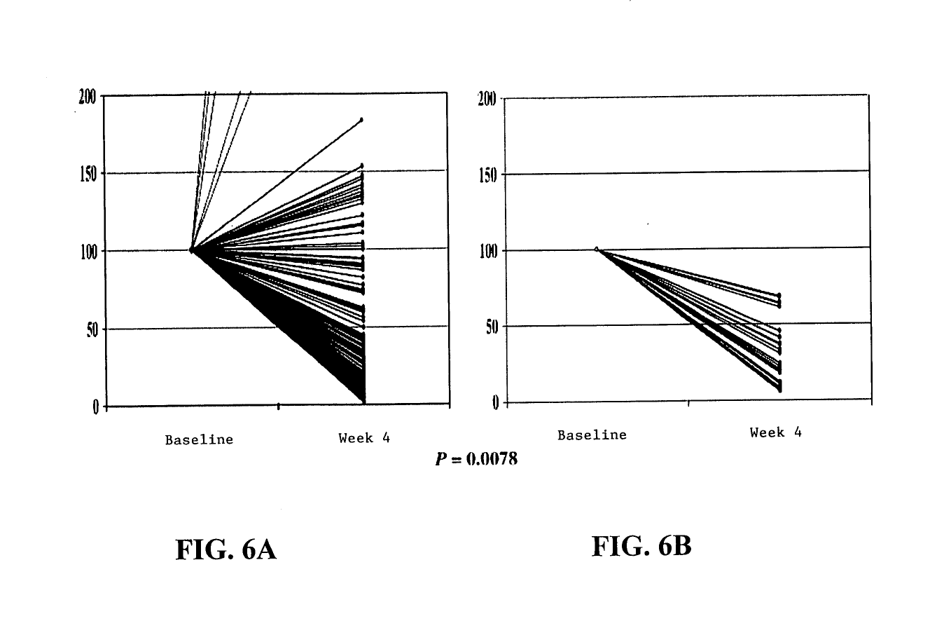

The frequencies of biological complete, partial and non-responders, according to the FCGR3A genotype, are shown in Table 10. Overall, there was a significant difference between genotypes (P=0.01). The best response was observed in V/V homozygotes and the worst response was found in F/F homozygotes. The most striking difference was observed when looking at the response rate (complete or partial) among V/V homozygotes when compared with other genotypes (FIG. 5). The median δCRP (relative value) was −80.1% (range, −31.0% to −94.8%) in V/V homozygotes vs. −63.2% (range, +1100% to −98.1%) in V/F and F/F patients together (P=0.0078) (FIGS. 6A and 6B). The median 6CRP (absolute value, normalized) was −8.0 (range, −1.0 to −119.0) in V/V patients vs. −6.6 (range, +107.0 to −58) in the F carrier group (P=0.05).

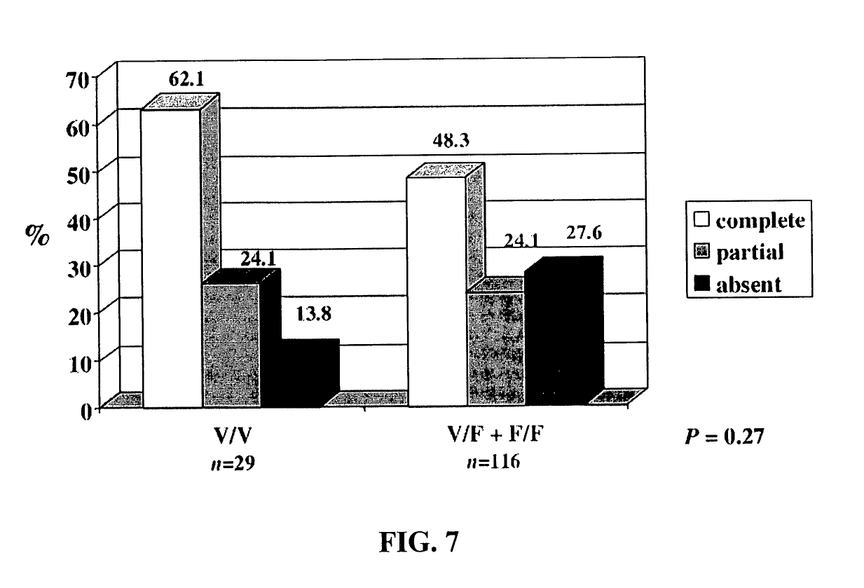

The frequencies of clinical complete, partial and non-responders, according to the FCGR3A genotype, in the sub-group of 145 patients (including 113 luminal and 32 fistulizing Crohn's disease) with elevated CRP before treatment, are shown in FIG. 7. Globally, there was no significant difference but, when comparing only the frequencies of non-responders with complete and partial responders grouped together, there was a trend towards a higher response rate in V/V patients (P=0.15), relative risk, 1.19; 95% CI, 0.99-1.43.

Multivariate Analyses

The logistic regression with clinical response as the dependent variable, on the whole group of patients for whom all clinical, demographic and biological data were available before treatment (n=189), identified the use of immunosuppressive drugs and an elevated CRP level (greater than twice the upper limit of the normal range) as factors predictive of response (P=0.003). The logistic regression with biological response as the dependent variable, on the sub-group of 145 patients with an elevated CRP before treatment, could not be performed because the FCGR3A genotype gave a complete separation of the patients (100% of responders amongst the V/V homozygotes). The logistic regression with clinical response as the dependent variable, on the sub-group of 145 patients with elevated CRP before treatment, selected the use of immunosuppressive drugs and the FCGR3A-158V/V genotype as factors predictive of response (P=0.003).

Discussion

Our results show a significant association between a biological response to infliximab, assessed by the variation in CRP level, and a single nucleotide polymorphism in the FCGR3A gene, which codes for a receptor for the Fc portion of IgG (FcγR) on natural killer cells and macrophages. There was also a trend towards an association between this polymorphism and a clinical response to infliximab.

Several arguments may explain why the FCGR3A effect was more prominent when a biological marker was considered (the CRP serum concentration), related to mucosal immuno-inflammatory processes, rather than the clinical response (adopting a CDAI decrease of 70 or 100 points). First, a clinical evaluation of the response to treatment is sometimes difficult in Crohn's disease and the correlation between clinical and biological parameters is poor 4 Cellier C, Sahmoud T, Froguel E, et al. Correlations between clinical activity, endoscopic severity, and biological parameters in colonic or ileocolonic Crohn's disease. A prospective multicenter study of 121 cases. Gut 1994; 35: 231-5.; D'Haens G, Van Deventer S, Van Hogezand R, et al. Endoscopic and histological healing with infliximab anti-tumor necrosis factor antibodies in Crohn's disease: a European multicenter trial. Gastroenterology 1999; 116: 1029-34). Second, the symptoms and clinical activity in Crohn's disease may result from a wide range of factors, and not only inflammation which is targeted by infliximab. Finally, certain non-responses to infliximab may be related to an absence of significant inflammation before treatment (Louis E, Vermeire S, Rutgeerts P, et al A positive response to infliximab in Crohn's disease: association with a higher systemic inflammation before treatment but not with −308 TNF gene polymorphism. Scand J Gastroenterol 2002; 37: 818-24), such as in clinical forms of Crohn's disease characterized by a predominance of irritable bowel syndrome, or post-surgical symptoms for which infliximab has no impact and the FCGR3A gene polymorphism no influence. Indeed, when the sub-group of patients with elevated CRP before treatment was selected, a trend towards an association between the FCGR3A gene polymorphism and a clinical response to infliximab was observed.

Similar to rituximab used in the treatment of follicular non-Hodgkin's lymphomas (Cartron G, Dacheux L, Salles G, et al. Therapeutic activity of humanized anti-CD20 monoclonal antibody and polymorphism in IgG Fc receptor FcγRIIIa gene. Blood 2002; 99: 754-8; Weng W K, Levy R. Two immunoglobulin G Fc receptor polymorphisms independently predict response to rituximab in patients with follicular lymphoma. J Clin Oncol 2003; 21: 3940-7) FCGR3A-158V/V homozygous patients developed a better response to infliximab, whereas sub-optimal responses were observed in F/F homozygotes. According to the association suggested by the clinical response to rituximab (Cartron G, Dacheux L, Salles G, et al Therapeutic activity of humanized anti-CD20 monoclonal antibody and polymorphism in IgG Fc receptor FcγRIIIa gene. Blood 2002; 99: 754-8; Weng W K, Levy R. Two immunoglobulin G Fc receptor polymorphisms independently predict response to rituximab in patients with follicular lymphoma. J Clin Oncol 2003; 21: 3940-7), V homozygosity confers a functional advantage, whereas the carriage of a single V allele in heterozygotes does not seem to be sufficient for a better response than in F homozygotes. This is the reason why the majority of the analyses were performed by grouping V/F and F/F patients together. The most striking difference concerned the proportion of biological non-responses: none in V/V patients vs. 30.2% in F carriers. This difference was also noted for the clinical response (although not statistically significant): in patients with elevated CRP before treatment, only 13.8% of V/V patients did not clinically respond vs. 27.6% of F carriers; in the whole group of patients, 17.1% of the V/V patients showed no response vs. 27.3% of F carriers. An analysis of a larger number of patients will be required to reach a more striking statistical significance concerning the association between the FCGR3A polymorphism and the clinical response to infliximab. According to our data, however, for a given patient, the predictive value of the FCGR3A genotype on the clinical response to infliximab in Crohn's disease is low.

The response to infliximab treatment is probably a complex phenomenon influenced by several parameters. In this model, the individual impact of each particular factor may not be very strong. So far, various clinical and demographic parameters, including the use of immunosuppressive drugs (Vermeire S, Louis E, Carbonez A, et al Demographic and clinical parameters influencing the short-term outcome of anti-tumor necrosis factor (infliximab) treatment in Crohn's disease. Am J Gastroenterol 2002; 97: 2357-63; Parsi M A, Achkar J P, Richardson S, et al. Predictors of response to infliximab in patients with Crohn's disease. Gastroenterology 2002; 123: 707-13) a young age (Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63) colonic location (Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63) non-smoking(Parsi M A, Achkar J P, Richardson S, et al. Predictors of response to infliximab in patients with Crohn's disease. Gastroenterology 2002; 123: 707-13) and elevated CRP (Louis E, Vermeire S, Rutgeerts P, et al. A positive response to infliximab in Crohn's disease: association with a higher systemic inflammation before treatment but not with −308 TNF gene polymorphism. Scand J Gastroenterol 2002; 37: 818-24) have been associated with a positive clinical response to infliximab, but no relevant pharmacogenetic association has been established. We studied all of these clinical and demographic parameters in a multivariate analysis with clinical response as the dependent variable. This analysis identified the use of immunosuppressive drugs and the presence of an elevated CRP level as factors predictive of a response to infliximab. In the sub-group of patients with an elevated CRP before treatment, the multivariate analysis identified the use of immunosuppressive drugs and the FCGR3A polymorphism as predictive factors. These results strengthen the case for a significant influence of the FCGR3A genotype on the response to infliximab, independent of other factors previously identified (Parsi M A, Achkar J P, Richardson S, et al Predictors of response to infliximab in patients with Crohn's disease. Gastroenterology 2002; 123: 707-13).

The FCGR3A gene studied encodes FcγRIIIa, a receptor for the Fc portion of IgG expressed on macrophages and natural killer cells and involved in antibody-dependent cell-mediated cytotoxicity. This mechanism has been identified in vitro using infliximab and cells bearing transmembrane TNF-α (Scallon B J, Moore M A, Trinh H, Knight D M, Ghrayeb J. Chimeric anti-TNF-alpha monoclonal antibody cA2 binds recombinant transmembrane TNF-alpha and activates immune effector functions. Cytokine 1995; 7: 251-9). The relative importance of antibody-dependent cell-mediated cytotoxicity in the therapeutic efficacy of infliximab in vivo, in addition to other mechanisms described in vitro, such as complement activation (Scallon B J, Moore M A, Trinh H, Knight D M, Ghrayeb J. Chimeric anti-TNF-alpha monoclonal antibody cA2 binds recombinant transmembrane TNF-alpha and activates immune effector functions. Cytokine 1995; 7: 251-9), apoptosis induction (Lügering A, Schmidt M, Lügering N, Pauels A G, Domschke W, Kucharzik T. Infliximab induces apoptosis in monocytes from patients with chronic active Crohn's disease by using a caspase-dependent pathway. Gastroenterology 2001; 121: 1145-57) or TNF-α neutralization (Siegel S A, Sheay D J, Nakada M T, et al. The mouse/human chimeric monoclonal antibody cA2 neutralizes TNF in vitro and protects transgenic mice from cachexia and TNF lethality in vivo. Cytokine 1995; 7: 15-25), is not known. However, the superiority of infliximab over etanercept in the treatment of Crohn's disease(Sandborn W, Hanauer S B, Katz S, et al Etanercept for active Crohn's disease: a randomised, double-blind, placebo-controlled trial. Gastroenterology 2001; 121: 1088-94) may be linked to a different ability to bind transmembrane TNF-α and, consequently, to induce the killing of activated TNF-α-positive mononuclear cells (Van den Brande J, Braat H, van den Brink G, et al. Infliximab but not etanercept induces apoptosis in lamina propria T-lymphocytes from patients with Crohn's disease. Gastroenterology 2003; 124: 1774-85). Indeed, although the capacity to bind soluble TNF-α was similar with the two drugs, only infliximab was able to induce apoptosis of activated lymphocytes in vitro (Van den Brande J, Braat H, van den Brink G, et al. Infliximab but not etanercept induces apoptosis in lamina propria T-lymphocytes from patients with Crohn's disease. Gastroenterology 2003; 124: 1774-85). In this in vitro model, neither complement-mediated nor cell-mediated lymphocyte lysis seemed to be involved. In another model, however, infliximab and etanercept also showed a different ability to mediate complement-dependent killing of TNF-α-expressing cells (Barone D, Krantz D, Maiori K, Moler K. Comparative analysis of the ability of etanercept and infliximab to lyse TNF-expressing cells in a complement-dependent fashion. Arthritis Rheum 1999;42(Suppl.): S90). In vivo, as demonstrated in some in vitro experiments (Scallon B J, Moore M A, Trinh H, Knight D M, Ghrayeb J. Chimeric anti-TNF-alpha monoclonal antibody cA2 binds recombinant transmembrane TNF-alpha and activates immune effector functions. Cytokine 1995; 7: 251-9; Shen C, Colpaert S, Maerten P, et al Infliximab induces death of human monocytes in vitro and in the THP-1-SCID-mouse model. Clin Immunol Suppl 2003; 1: S142) cell-mediated lysis, as a consequence of infliximab binding to transmembrane TNF-α, may further contribute to the efficacy of infliximab. The functionally significant FCGR3A-158 polymorphism may thus have an impact on infliximab efficacy by influencing its ability to kill mucosal mononuclear cells through antibody-dependent cell-mediated cytotoxicity, and thus may influence the rate of sustained response measured 4 weeks after treatment. This suggests that antibody-dependent cell-mediated cytotoxicity is one of the mechanisms involved in the action of infliximab, and that V/V patients are more likely than F carriers to have at least a biological response through this mechanism. However, further genetic studies (excluding linkage disequilibrium) and in vitro experiments will be required to demonstrate these hypotheses.

In conclusion, our results show, for the first time, a relevant pharmacogenetic association for infliximab in Crohn's disease. They suggest a role for FcγR and probably antibody-dependent cell-mediated cytotoxicity amongst the mechanisms of action of infliximab in Crohn's disease. Finally, they also emphasize the need for a biological, and not only clinical, assessment of response in Crohn's disease when studying new drugs, especially in pharmacogenetic investigations.

### REFERENCES

1. Maloney D G, Liles T M, Czerwinski D K, et al.: Phase I clinical trial using escalating single-dose infusion of chimeric anti-CD20 monoclonal antibody (IDEC-C2B8) in patients with recurrent B-cell lymphoma. Blood. 1994;84:2457-2466.
2. McLaughlin P, Grillo-Lopez A J, Link B K, et al.: Rituximab chimeric anti-CD20 monoclonal antibody therapy for relapsed indolent lymphoma: half of patients respond to a four-dose treatment program. J Clin Oncol. 1998;16:2825-2833.
3. Maloney D G, Grillo-Lopez A J, White C A, et al.: IDEC-C2B8 (Rituximab) anti-CD20 monoclonal antibody therapy in patients with relapsed low-grade non-Hodgkin's lymphoma. Blood. 1997;90:2188-2195.
4. Hainsworth J D, Burris H A, 3rd, Morrissey L H, et al: Rituximab monoclonal antibody as initial systemic therapy for patients with low-grade non-Hodgkin lymphoma. Blood. 2000;95:3052-3056.
5. Colombat P, Salles G, Brousse N, et al.: Rituximab (anti-CD20 monoclonal antibody) as first-line therapy of follicular lymphoma patients with low tumor burden: clinical and molecular evaluation. Blood. 2001;97:101-106.
6. Coiffier B, Haioun C, Ketterer N, et al: Rituximab (anti-CD20 monoclonal antibody) for the treatment of patients with relapsing or refractory aggressive lymphoma: a multicenter phase II study. Blood. 1998;92:1927-1932.
7. Foran J M, Rohatiner A Z, Cunningham D, et al: European phase II study of rituximab (chimeric anti-CD20 monoclonal antibody) for patients with newly diagnosed mantle-cell lymphoma and previously treated mantle-cell lymphoma, immunocytoma, and small B-cell lymphocytic lymphoma. J Clin Oncol. 2000;18:317-324.
8. Anderson D R, Grillo-Lopez A, Varns C, Chambers K S, Hanna N: Targeted anti-cancer therapy using rituximab, a chimaeric anti-CD20 antibody (IDEC-C2B8) in the treatment of non-Hodgkin's B-cell lymphoma. Biochem Soc Trans. 1997;25:705-708.
9. Vose J, Link B, Grossbard M, et al: Phase II study of rituximab in combination with CHOP chemotherapy in patients with preiously untreated intermediate or high-grade non-Hodgkin's lymphoma (NHL). Ann Oncol. 1999;10:58.
10. Berinstein N L, Grillo-Lopez A J, White C A, et al: Association of serum Rituximab (IDEC-C2B8) concentration and anti-tumor response in the treatment of recurrent low-grade or follicular non-Hodgkin's lymphoma. Ann Oncol. 1998;9:995-1001.
11. Harjunpaa A, Junnikkala S, Meri S: Rituximab (anti-CD20) therapy of B-cell lymphomas: direct complement killing is superior to cellular effector mechanisms. Scand J. Immunol. 2000;51:634-641.
12. Reff M E, Carner K, Chambers K S, et al.: Depletion of B cells in vivo by a chimeric mouse human monoclonal antibody to CD20. Blood. 1994;83:435-445.
13. Idusogie E E, Presta L G, Gazzano-Santoro H, et al: Mapping of the C1q binding site on rituxan, a chimeric antibody with a human IgG 1 Fc. J. Immunol. 2000;164:4178-4184.
14. Golay J, Zaffaroni L, Vaccari T, et al: Biologic response of B lymphoma cells to anti-CD20 monoclonal antibody rituximab in vitro: CD55 and CD59 regulate complement-mediated cell lysis. Blood. 2000;95:3900-3908.
15. Shan D, Ledbetter J A, Press OW: Apoptosis of malignant human B cells by ligation of CD20 with monoclonal antibodies. Blood. 1998;91:1644-1652.
16. Shan D, Ledbetter J A, Press OW: Signaling events involved in anti-CD20-induced apoptosis of malignant human B cells. Cancer Immunol Immunother. 2000;48:673-683.
17. Hooijberg E, Sein J J, van den Berk P C, et al: Eradication of large human B cell tumors in nude mice with unconjugated CD20 monoclonal antibodies and interleukin 2. Cancer Res. 1995;55:2627-2634.
18. Funakoshi S, Longo D L, Murphy W J: Differential in vitro and in vivo antitumor effects mediated by anti-CD40 and anti-CD20 monoclonal antibodies against human B-cell lymphomas. J Immunother Emphasis Tumor Immunol. 1996;19:93-101.
19. Clynes R A, Towers T L, Presta L G, Ravetch J V: Inhibitory Fc receptors modulate in vivo cytoxicity against tumor targets. Nat Med. 2000;6:443-446.
20. Fijen C A, Bredius R G, Kuijper E J, et al: The role of Fcγ receptor polymorphisms and C3 in the immune defence against Neisseria meningitidis in complement-deficient individuals. Clin Exp Immunol. 2000;120:338-345.
21. Dijstelbloem H M, Scheepers R H, Oost W W, et al: Fcγ receptor polymorphisms in Wegener's granulomatosis: risk factors for disease relapse. Arthritis Rheum. 1999;42:1823-1827.
22. Myhr K M, Raknes G, Nyland H, Vedeler C: Immunoglobulin G Fc-receptor (FcγR) IIA and IIIB polymorphisms related to disability in MS. Neurology. 1999;52:1771-1776.
23. Koene H R, Kleijer M, Algra J, Roos D, von dem Borne A E, de Haas M: Fc γRIIIa-158V/F polymorphism influences the binding of IgG by natural killer cell Fc γRIIa, independently of the Fc γRIIIa-48L/R/H phenotype. Blood. 1997;90:1109-1114.
24. Wu J, Edberg J C, Redecha P B, et al.: A novel polymorphism of FcγRIIIa (CD16) alters receptor function and predisposes to autoimmune disease. J Clin Invest. 1997;100:1059-1070.
25. Sondermann P, Huber R, Oosthuizen V, Jacob U: The 3.2-A crystal structure of the human IgG1 Fc fragment-Fc γRIII complex. Nature. 2000;406:267-273.
26. Harris N L, Jaffe E S, Stein H, et al.: A revised European-American classification of lymphoid neoplasms: a proposal from the International Lymphoma Study Group. Blood. 1994;84:1361-1392.
27. Brice P, Bastion Y, Lepage E, et al.: Comparison in low-tumor-burden follicular lymphomas between an initial no-treatment policy, prednimustine, or interferon alfa: a randomized study from the Groupe d'Etude des Lymphomes Folliculaires. Groupe d'Etude des Lymphomes de l'Adulte. J Clin Oncol. 1997;15:1110-1117.
28. Cheson B D, Horning S J, Coiffier B, et al.: Report of an international workshop to standardize response criteria for non-Hodgkin's lymphomas. NCI Sponsored International Working. J Clin Oncol. 1999;17:1244.
29. Liang X M, Arepally G, Poncz M, McKenzie S E: Rapid detection of the Fc γRIIA-H/R 131 ligand-binding polymorphism using an allele-specific restriction enzyme digestion (ASRED). J Immunol Methods. 1996;199:55-59.
30. Kaplan E, Meier P: Nonparametric estimation from incomplete observations. J Am Stat Assoc. 1958;53:457-481.
31. de Haas M, Koene H R, Kleijer M, et al.: A triallelic Fc γ receptor type IIIA polymorphism influences the binding of human IgG by NK cell Fc γ RIIIa. J. Immunol. 1996;156:3948-3955.
32. Peltz G A, Grundy H O, Lebo R V, Yssel H, Barsh G S, Moore K W: Human Fc γ RIII: cloning, expression, and identification of the chromosomal locus of two Fc receptors for IgG. Proc Nad Acad Sci USA. 1989;86:1013-1017.
33. Schnackenberg L, Flesch B K, Neppert J: Linkage disequilibria between Duffy blood groups, Fc γ IIa and Fc γ IIIb allotypes. Exp Clin Immunogenet. 1997;14:235-242.
34. Shields R L, Namenuk A K, Hong K, et al.: High resolution mapping of the binding site on human IgG1 for Fc γ RI, Fc γ RII, Fc γ RIII, and FcRn and design of IgG1 variants with improved binding to the Fc γ R. J Biol. Chem. 2001;276:6591-6604.
35. Leppers-van de Straat F G, van der Pol W, Jansen M D, et al.: A novel PCR-based method for direct Fcγ receptor IIIa (CD16) allotyping. J Immunol Methods. 2000;242:127-132.
36. Lehrnbecher T, Foster C B, Zhu S, et al.: Variant genotypes of the low-affinity Fcγ receptors in two control populations and a review of low-affinity Fcγ receptor polymorphisms in control and disease populations. Blood. 1999;94:4220-4232.
37. Knight D M, Trinh H, Le J, et al. Construction and initial characterization of a human mouse chimeric antibody. Mol Immunol 1993; 30: 1443-53.
38. Targan S, Hanauer S, van Deventer S J H, et al. A short term study of chimeric antibody cA2 to tumor necrosis factor alpha for Crohn's disease. N Engl J Med 1997; 337: 1029-35.
39. Present D H, Rutgeerts P, Targan S, et al Infliximab for the treatment of fistulas in patients with Crohn's disease. N Engl J Med 1999; 340:1398-405.
40. Cohen R D, Tsang J F, Hanauer S B. Infliximab in Crohn's disease: first anniversary clinical experience. Am J Gastroenterol 2000; 95: 3469-77.
41. Farrell R J, Shah S A, Lodhavia P J, et al. Clinical experience with infliximab therapy in 100 patients with Crohn's disease. Am J Gastroenterol 2000; 95: 3490-7.
42. Vermeire S, Louis E, Carbonez A, et al. Demographic and clinical parameters influencing the short-term outcome of anti-tumor necrosis factor (infliximab) treatment in Crohn's disease. Am J Gastroenterol 2002; 97: 2357-63.
43. Parsi M A, Achkar J P, Richardson S, et al Predictors of response to infliximab in patients with Crohn's disease. Gastroenterology 2002; 123: 707-13.
44. Taylor K D, Plevy S E, Yang H, et al. Anca pattern and LTA haplotype relationship to clinical responses to anti-TNF antibody treatment in Crohn's disease. Gastroenterology 2001; 120: 1347-55.
45. Louis E, Verneire S, Rutgeerts P, et al. A positive response to infliximab in Crohn's disease: association with a higher systemic inflammation before treatment but not with -308 TNF gene polymorphism. Scand J Gastroenterol 2002; 37: 818-24.
46. Vermeire S, Louis E, Rutgeerts P, et al. NOD2/CARD15 does not influence response to infliximab in Crohn's disease. Gastroenterology 2002; 123: 106-11.
47. Lügering A, Schmidt M, Lügering N, Pauels A G, Domschke W, Kucharzik T. Infliximab induces apoptosis in monocytes from patients with chronic active Crohn's disease by using a caspase-dependent pathway. Gastroenterology 2001; 121: 1145-57.
48. Scallon B J, Moore M A, Trinh H, Knight D M, Ghrayeb J. Chimeric anti-TNF-alpha monoclonal antibody cA2 binds recombinant transmembrane TNF-alpha and activates immune effector functions. Cytokine 1995; 7: 251-9.
49. Cartron G, Dacheux L, Salles G, et al. Therapeutic activity of humanized anti-CD20 monoclonal antibody and polymorphism in IgG Fc receptor FcγRIIa gene. Blood 2002; 99: 754-8.
50. Weng WK, Levy R. Two immunoglobulin G Fc receptor polymorphisms independently predict response to rituximab in patients with follicular lymphoma. J Clin Oncol 2003; 21: 3940-7.
51. Dall'Ozzo S, Andres C, Bardos P, Watier H, Thibault G. Rapid single-step FCGR3A genotyping based on SYBR Green I fluorescence in real-time multiplex allele-specific PCR. J Immunol Methods 2003; 277: 185-92.
52. Solberg HE. Approved recommendation on the theory of reference values. Part 5. Statistical treatment of collected reference values. Determination of reference limits. J Clin Chem Clin Biochem 1987; 25: 645-56.
53. Cellier C, Sahmoud T, Froguel E, et al Correlations between clinical activity, endoscopic severity, and biological parameters in colonic or ileocolonic Crohn's disease. A prospective multicentre study of 121 cases. Gut 1994; 35: 231-5.
54. D'Haens G, Van Deventer S, Van Hogezand R, et al Endoscopic and histological healing with infliximab anti-tumor necrosis factor antibodies in Crohn's disease: a European multicenter trial. Gastroenterology 1999; 116: 1029-34.
55. Siegel S A, Sheay D J, Nakada M T, et al. The mouse/human chimeric monoclonal antibody cA2 neutralizes TNF in vitro and protects transgenic mice from cachexia and TNF lethality in vivo. Cytokine 1995; 7: 15-25.
56. Sandborn A, Hanauer S B, Katz S, et al. Etanercept for active Crohn's disease: a randomised, double-blind, placebo-controlled trial. Gastroenterology 2001; 121: 1088-94.
57. Van den Brande J, Braat H, van den Brink G, et al. Infliximab but not etanercept induces apoptosis in lamina propria T-lymphocytes from patients with Crohn's disease. Gastroenterology 2003; 124: 1774-85.
58. Barone D, Krantz D, Maiori K, Moler K. Comparative analysis of the ability of etanercept and infliximab to lyse TNF-expressing cells in a complement-dependent fashion. Arthritis Rheum 1999; 42(Suppl.): S90.

59. Shen C, Colpaert S, Maerten P, et al. Infliximab induces death of human monocytes in vitro and in the THP-1-SCID-mouse model. Clin Immunol Suppl 2003; 1: S142.

TABLE 1
CHARACTERISTICS OF PATIENTS ACCORDING TO THE
FCGR3A-158V/F POLYMORPHISM.

| | FCGR3A- | FCGR3A- | FCGR3A- | |
|---|---|---|---|---|
| | 158VV | 158VF | 158FF | p* |
| n (%) | 10 (20%) | 22 (45%) | 17 (35%) | |
| Sex | | | | |
| M | 3 | 12 | 10 | ns |
| F | 7 | 10 | 7 | |
| Disease stage | | | | |
| II-III | 3 | 6 | 6 | ns |
| IV | 7 | 16 | 11 | |
| Bone marrow involvement | | | | |
| yes | 7 | 16 | 9 | ns |
| no | 3 | 6 | 8 | |
| Extra-nodal sites involved | | | | |
| <2 | 8 | 20 | 13 | ns |
| ≧2 | 2 | 2 | 4 | |
| BCL2-JH rearrangement | 8 | 12 | 11 | ns |
| in peripheral blood | | | | |
| BCL2-JH rearrangement | 7 | 12 | 11 | ns |
| in bone marrow | | | | |

*Statistical comparisons of the three groups of homozygous FCGR3A-158V patients vs. FCGR3A-158F carriers and of homozygous FCGR3A-158F patients against FCGR3A-158V carriers.

TABLE 2
CLINICAL RESPONSE TO RITUXIMAB BY FCGR3A-158V/F
POLYMORPHISM.

| | FCGR3A- | FCGR3A- | |
|---|---|---|---|
| | 158VV | 158F carriers | p* |
| Clinical response at M2 | | | |
| Objective response | 10 (100%) | 26 (67%) | 0.03 |
| complete remission | 3 | 7 | |
| complete remission unconfirmed | 1 | 2 | |
| partial response | 6 | 17 | |
| No response | 0 (0%) | 13 (33%) | |
| no change | 0 | 10 | |
| progressive disease | 0 | 3 | |
| Clinical response at M12 | | | |
| Objective response | 9 (90%) | 20 (51%) | 0.03 |
| complete remission | 6 | 11 | |
| complete remission unconfirmed | 1 | 2 | |
| partial response | 2 | 7 | |
| No response | 1 (10%) | 19 (49%) | |
| no change | 0 | 2 | |
| progressive disease | 1 | 17 | |

*Statistical comparison of homozygous FCGR3A-158V patients against FCGR3A-158F carriers. Data concerning the three genotype subgroups are given in the text.

TABLE 3
MOLECULAR RESPONSE TO RITUXIMAB AT M2 AND AT
M12 BY THE FCGR3A-158V/F POLYMORPHISM.

| | FCGR3A- | FCGR3A-158F | |
|---|---|---|---|
| | 158VV | carriers | p |
| Molecular response at M2 | | | ns |
| Cleaning of BCL2-JH rearrangement | 3 | 5 | |
| Persistent BCL2-JH rearrangement | 3 | 14 | |
| Molecular response at M12 | | | 0.03 |
| Cleaning of BCL2-JH rearrangement | 5 | 5 | |
| Persistent BCL2-JH rearrangement | 1 | 12 | |

TABLE 4
Therapeutic Antibodies.

| Ab specificity | DCI | Commercial name | Typical Indications |
|---|---|---|---|
| Anti-CD20 | rituximab | MabThera ®, Rituxan ® | LNH B |
| Anti-CD52 | alemtuzumab | CAMPATH-1H ® | LLC, allograft |
| Anti-CD33 | | Zamyl ™ | Acute myeloid Leukemia |
| Anti-HLA-DR | | Remitogen ™ | LNH B |
| Anti-CD22 | epratuzumab | LymphoCide ™ | LNH B |
| Anti-erbB2 | trastuzumab | Herceptin ®, | Metastatic breast cancer |
| (HER-2/neu) | | | |
| Anti-EGFR | cetuximab | | ORL and colorectal Cancers |
| (HER-1, | | | |
| erbB1) | | | |
| Anti-MUC-1 | | Therex ® | Breast and epithelial cancers |
| Anti-CEA | labetuzumab | CEA-Cide ™ | |
| Anti-αVβ3 | | Vitaxin | Cancers (anti-angiogenic) |
| | | | Cancers (anti-angiogenic) |
| Anti-KDR | palivizumab | Synagis ® | Viral diseases |
| (VEGFR2) | | | |
| anti-VRS | | | |
| fusion protein | | | |
| Idem | | Numax ™ | Idem |
| CMV | sevirumab | Protovir | CMV Infection |
| HBs | tuvirumab | Ostavir ™ | Hepatitis B |
| Anti-CD25 | basiliximab | Simulect ® | Prevention/treatment allograft |
| | | | rejection |
| Anti-CD25 | daclizumab | Zénapax ® | Prevention/treatment allograft |
| | | | rejection |
| anti-TNF-α | infliximab | Remicade ™ | Crohn's disease, polyarthrite |
| | | | rheumatoid |
| anti-IgE | omalizumab | Xolair ™ | Asthma |
| anti-integrin | efalizumab | Xanelim ™ | psoriasis |
| αL (CD11a, | | | |
| LFA-1) | | | |
| anti-CD4 | keliximab | | |
| anti-CD2 | siplizumab | | |
| Anti-CD64 | | | anaemia |
| anti-CD147 | | | GvH |
| anti-integrin | natalizumab | Antegren ® | Sclerosis, Crohn's Disease |
| α4 (α4β1-α4β7) | | | |
| Anti-integrin | | | Crohn's Disease, RCH |
| β7 | | | |
| anti-CD4* | clenoliximab | | |
| anti-TNF-α | D2E7 | | Crohn's Disease |
| anti-TNF-α | CDP571 | | Crohn's Disease; |
| | | | rheumatoid arthritis |

TABLE 5
ANTIBODIES FOR INFLAMMATORY DISEASES AND AUTOIMMUNE DISEASES

| Antibody Name | Target Antigen | Product Type | Isotype | Sponsors | Indication |
|---|---|---|---|---|---|
| 5G1.1 | Complement (C5) | Humanised | IgG | Alexion Pharm Inc | Rheumatoid Arthritis |
| 5G1.1 | Complement (C5) | Humanised | IgG | Alexion Pharm Inc | SLE |
| 5G1.1 | Complement (C5) | Humanised | IgG | Alexion Pharm Inc | Nephritis |
| 5G1.1-SC | Complement (C5) | Humanised | ScFv | Alexion Pharm Inc | Cardiopulmano Bypass |
| 5G1.1-SC | Complement (C5) | Humanised | ScFv | Alexion Pharm Inc | Myocardial Infarction |
| 5G1.1-SC | Complement (C5) | Humanised | ScFv | Alexion Pharm Inc | Angioplasty |
| ABX-CBL | CBL | Human | | Abgenix Inc | GvHD |
| ABX-CBL | CD147 | Murine | IgG | Abgenix Inc | Allograft rejection |
| ABX-IL8 | IL-8 | Human | IgG2 | Abgenix Inc | Psoriasis |
| Antegren | VLA-4 | Humanised | IgG | Athena/Elan | Multiple Sclerosis |
| Anti-CD11a | CD11a | Humanised | IgG1 | Genentech Inc/Xoma | Psoriasis |
| Anti-CD18 | CD18 | Humanised | Fab′2 | Genentech Inc | Myocardial infarction |
| Anti-LFA1 | CD18 | Murine | Fab′2 | Pasteur-Merieux/ | Allograft rejection |
| | | | | Immunotech | |
| Antova | CD40L | Humanised | IgG | Biogen | Allograft rejection |
| Antova | CD40L | Humanised | IgG | Biogen | SLE |
| BTI-322 | CD2 | Rat | IgG | Medimmune Inc | GvHD, Psoriasis |
| CDP571 | TNF-alpha | Humanised | IgG4 | Celltech | Crohn's |
| CDP571 | TNF-alpha | Humanised | IgG4 | Celltech | Rheumatoid Arthritis |
| CDP850 | E-selectin | Humanised | | Celltech | Psoriasis |
| Corsevin M | Fact VII | Chimeric | | Centocor | Anticoagulant |
| D2E7 | TNF-alpha | Human | | CAT/BASF | Rheumatoid Arthritis |
| Hu23F2G | CD11/18 | Humanised | | ICOS Pharm Inc | Multiple Sclerosis |
| Hu23F2G | CD11/18 | Humanised | IgG | ICOS Pharm Inc | Stroke |
| IC14 | CD14 | ? | | ICOS Pharm Inc | Toxic shock |
| ICM3 | ICAM-3 | Humanised | | ICOS Pharm Inc | Psoriasis |
| IDEC-114 | CD80 | Primatised | | IDEC | Psoriasis |
| | | | | Pharm/Mitsubishi | |
| IDEC-131 | CD40L | Humanised | | IDEC Pharm/Eisai | SLE |
| IDEC-131 | CD40L | Humanised | | IDEC Pharm/Eisai | Multiple Sclerosis |
| IDEC-151 | CD4 | Primatised | IgG1 | IDEC | Rheumatoid Arthritis |
| | | | | Pharm/GlaxoSmithKline | |
| IDEC-152 | CD23 | Primatised | | IDEC Pharm | Asthma/Allergy |
| Infliximab | TNF-alpha | Chimeric | IgG1 | Centocor | Rheumatoid Arthritis |
| Infliximab | TNF-alpha | Chimeric | IgG1 | Centocor | Crohn's |
| LDP-01 | Beta2-integrin | Humanised | IgG | Millennium Inc | Stroke |
| | | | | (LeukoSite Inc.) | |
| LDP-01 | Beta2-integrin | Humanised | IgG | Millennium Inc | Allograft rejection |
| | | | | (LeukoSite Inc.) | |
| LDP-02 | alpha4beta7 | Humanised | | Millennium Inc | Ulcerative Colitis |
| | | | | (LeukoSite Inc.) | |
| MAK-195F | TNF alpha | Murine | Fab′2 | Knoll Pharm, BASF | Toxic shock |
| MDX-33 | CD64 (FcR) | Human | | Medarex/Centeon | Autoimmune haematogical |
| | | | | | disorders |
| MDX-CD4 | CD4 | Human | IgG | Medarex/Eisai/ | Rheumatoid Arthritis |
| | | | | Genmab | |
| MEDI-507 | CD2 | Humanised | | Medimmune Inc | Psoriasis |
| MEDI-507 | CD2 | Humanised | | Medimmune Inc | GvHD |
| OKT4A | CD4 | Humanised | IgG | Ortho Biotech | Allograft rejection |
| OrthoClone | CD4 | Humanised | IgG | Ortho Biotech | Autoimmune disease |
| OKT4A | | | | | |
| Orthoclone/ | CD3 | Murine | mIgG2a | Ortho Biotech | Allograft rejection |
| anti-CD3 | | | | | |
| OKT3 | | | | | |
| RepPro/ | gpIIbIIIa | Chimeric | Fab | Centocor/Lilly | Complications of coronary |
| Abciximab | | | | | angioplasty |
| rhuMab-E25 | IgE | Humanised | IgG1 | Genentech/Novartis/ | Asthma/Allergy |
| | | | | Tanox Biosystems | |
| SB-240563 | IL5 | Humanised | | GlaxoSmithKline | Asthma/Allergy |
| SB-240683 | IL-4 | Humanised | | GlaxoSmithKline | Asthma/Allergy |
| SCH55700 | IL-5 | Humanised | | Celltech/Schering | Asthma/Allergy |
| Simulect | CD25 | Chimeric | IgG1 | Novartis Pharm | Allograft rejection |
| SMART | CD3 | Humanised | | Protein Design Lab | Autoimmune disease |
| a-CD3 | | | | | |
| SMART | CD3 | Humanised | | Protein Design Lab | Allograft rejection |
| a-CD3 | | | | | |
| SMART | CD3 | Humanised | IgG | Protein Design Lab | Psoriasis |
| a-CD3 | | | | | |
| Zenapax | CD25 | Humanised | IgG1 | Protein Design | Allograft rejection |
| | | | | Lab/Hoffman- | |
| | | | | La Roche | |

TABLE 6
Antibodies for Autoimmune Disorders

| Antibody | Indication | Target Antigen |
|---|---|---|
| ABX-RB2 | | antibody to CBL antigen on T cells, B |
| | | cells and NK cells |
| | | fully human antibody from the |
| | | Xenomouse |
| IL1-ra | rheumatoid arthritis | recombinant anti-inflammatory protein |
| sTNF-RI | chronic inflammatory disease | soluble tumor necrosis factor a - receptor |
| | rheumatoid arthritis | type I |
| | | blocks TNF action |
| 5c8 (Anti CD-40 | Phase II trials were halted in Oct. 99 | CD-40 |
| ligand antibody) | examine “adverse events” | |
| IDEC 131 | systemic lupus erythyematous (SLE) | anti CD40 |
| | | humanized |
| IDEC 151 | rheumatoid arthritis | primatized; anti-CD4 |
| IDEC 152 | asthma | primatized; anti-CD23 |
| IDEC 114 | psoriasis | primatized anti-CD80 |
| MEDI-507 | rheumatoid arthritis; multiple sclerosis | anti-CD2 |
| | Crohn's disease | |
| | psoriasis | |
| LDP-02 (anti-b7 mAb) | inflammatory bowel disease | a4b7 integrin receptor on white blood |
| | Chron's disease | cells (leukocytes) |
| | ulcerative colitis | |
| SMART Anti-Gamma | autoimmune disorders | Anti-Gamma Interferon |
| Interferon antibody | | |
| Verteportin | rheumatoid arthritis | |
| Thalomid | leprosy - approved for market Chron's | inhibitor of tumor necrosis factor alpha |
| (thalidomide) | disease | (TNF alpha) |
| | rheumatoid arthritis | |
| SelCIDs (selective | | highly specific |
| cytokine inhibitory | | inhibitors of phosphodiesterase type 4 |
| drugs) | | enzyme (PDE-4) |
| | | increases levels of cAMP (cyclic |
| | | adenosine monophosphate) |
| | | activates protein kinase A (PKA) |
| | | blocks transcription factor NK-kB |
| | | prevents transcription of TNF-a gene |
| | | decreases production of TNF-a |
| IMiDs | general autoimmune disorders | structural analogues of thalidomideinhibit |
| (immunomodulatory | | TNF-a |
| drugs) | | |
| MDX-33 | blood disorders caused by autoimmune | monoclonal antibody against FcRI |
| | reactions | receptors |
| | Idiopathic Thrombocytopenia Purpurea | |
| | (ITP) | |
| | autoimmune hemolytic anemia | |
| MDX-CD4 | treat rheumatoid arthritis and other | monoclonal antibody against CD4 |
| | autoimmunity | receptor molecule |
| VX-497 | autoimmune disorders | inhibitor of inosine monophosphate |
| | multiple sclerosis | dehydrogenase |
| | rheumatoid arthritis | (enzyme needed to make new RNA and |
| | inflammatory bowel disease | DNA |
| | lupus | used in production of nucleotides |
| | psoriasis | needed for lymphocyte proliferation) |
| VX-740 | rheumatoid arthritis | inhibitor of ICE |
| | | interleukin-1 beta (converting enzyme |
| | | controls pathways leading to aggressive |
| | | immune response |
| | | regulates cytokines) |
| VX-745 | specific to inflammation | inhibitor of P38MAP kinase |
| | involved in chemical signalling of | mitogen activated protein kinase |
| | immune response | |
| | onset and progression of inflammation | |
| Enbrel (etanercept) | | targets TNF (tumor necrosis factor) |
| IL-8 | | fully human MAB against IL-8 |
| | | (interleukin 8) |
| | | (blocks IL-8 |
| | | blocks inflammatory response) |
| 5G1.1 | rheumatoid arthritis | a C5 complement inhibitor |
| | pemphigoid (dangerous skin rash) | |
| | psoriasis | |
| | lupus | |
| Apogen MP4 | | recombinant antigen |
| | | selectively destroys disease associated T- |
| | | cells |
| | | induces apoptosis |
| | | T-cells eliminated by programmed cell |
| | | death |
| | | no longer attack body's own cells |
| | | specific apogens target specific T-cells |

| Company Rankings | Product | Development Stage |
|---|---|---|
| Immunex | Enbrel | on market |
| Amgen | IL1-ra, sTNF-RI | Phase II/III |
| Abgenix | AGX-RB2, IL-8 | preclinical, Phase I |
| Alexion | 5G1.1, Apogen MP4 | Phase II, preclinical |
| Biogen | 5c8 | Phase II (halted) |
| IDEC | 131, 151, 152, 114 | Phase I and II |
| MedImmune | MEDI 507 | Phase I/II |
| Millennium | LDP-02, | Phase II |
| Protein Design Labs | Anti-Gamma Interferon | preclinical |
| Medarex | MDX-33, MDX-CD4 | Phase II, Phase I |
| QLT PhotoTherapeutics | Verteportin | Phase I |
| Celegene | Thalomid, SelCIDs, IMiDs | on market, preclinical |
| Vertex | VX-497, VX-740, VX-745 | Phase II, Phase II, Phase II |

TABLE 7
MONOCLONAL ANTIBODIES FOR CANCER THERAPY

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic cancers | tumor antigen MUC1 |
| Antisoma | Theragyn (pemtumornabytrrium-90) | ovarian cancer | PEM antigen |
| | Therex | breast cancer | PEM antigen |
| Boehringer Ingelheim | bivatuzumab | head & neck cancer | CD44 |
| Centocor/J&J | Panorex | Colorectal cancer | 17-1A |
| | ReoPro | PTCA | gp IIIb/IIIa |
| | ReoPro | Acute MI | gp IIIb/IIIa |
| | ReoPro | Ischemic stroke | gp IIIb/IIIa |
| Corixa | Bexocar | NHL | CD20 |
| CRC Technology | MAb, idiotypic 105AD7 | colorectal cancer vaccine | gp72 |
| Crucell | Anti-EpCAM | cancer | Ep-CAM |
| Cytoclonal | MAb, lung cancer | non-small cell lung | NA |
| | | cancer | |
| Genentech | Herceptin | metastatic breast cancer | HER-2 |
| | Herceptin | early stage breast cancer | HER-2 |
| | Rituxan | Relapsed/refractory low- | CD20 |
| | | grade or follicular NHL | |
| | Rituxan | intermediate & high- | CD20 |
| | | grade NHL | |
| | MAb-VEGF | NSCLC, metastatic | VEGF |
| | MAb-VEGF | Colorectal cancer, | VEGF |
| | | metastatic | |
| | AMD Fab | age-related macular | CD18 |
| | | degeneration | |
| | E-26 (2^{nd} gen. IgE) | allergic asthma & rhinitis | IgE |
| IDEC | Zevalin (Rituxan + yttrium-90) | low grade of follicular, | CD20 |
| | | relapsed or refractory, | |
| | | CD20-positive, B-cell | |
| | | NHL and Rituximab- | |
| | | refractory NHL | |
| ImClone | Cetuximab + innotecan | refractory colorectal | EGF receptor |
| | | carcinoma | |
| | Cetuximab + cisplatin & radiation | newly diagnosed or | EGF receptor |
| | | recurrent head & neck | |
| | | cancer | |
| | Cetuximab + gemcitabine | newly diagnosed | EGF receptor |
| | | metastatic pancreatic | |
| | | carcinoma | |
| | Cetuximab + cisplatin + 5FU or | recurrent of metastatic | EGF receptor |
| | Taxol | head & neck cancer | |
| | Cetuximab + carboplatin + paclitaxel | newly diagnosed non- | EGF receptor |
| | | small cell lung carcinoma | |
| | Cetuximab + cisplatin | head & neck cancer | EGF receptor |
| | | (extensive incurable | |
| | | local-regional disease & | |
| | | distant metasteses) | |
| | Cetuximab + radiation | locally advanced head & | EGF receptor |
| | | neck carcinoma | |
| | BEC2 + Bacillus Calmette Guerin | small cell lung carcinoma | mimics ganglioside GD3 |
| | BEC2 + Bacillus Calmette Guerin | melanoma | mimics ganglioside GD3 |
| | IMC-1C11 | colorectal cancer with | VEGF-receptor |
| | | liver metasteses | |
| ImmonoGen | nuC242-DMI | Colorectal, gastric, and | nuC242 |
| | | pancreatic cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid tumors | CEA |
| | CEA-Cide Y-90 | metastatic solid tumors | CEA |
| | CEA-Scan (Tc-99m-labeled | colorectal cancer | CEA |
| | arcitumomab) | (radioimaging) | |
| | CEA-Scan (Tc-99m-labeled | Breast cancer | CEA |
| | arcitumomab) | (radioimaging) | |
| | CEA-Scan (Tc-99m-labeled | lung cancer | CEA |
| | arcitumomab) | (radioimaging) | |
| | CEA-Scan (Tc-99m-labeled | intraoperative tumors | CEA |
| | arcitumomab) | (radio imaging) | |
| | LeukoScan (Tc-99m-labeled | soft tissue infection | CEA |
| | sulesomab) | (radioimaging) | |
| | LymphoScan (Tc-99m-labeled) | lymphomas | CD22 |
| | | (radioimaging) | |
| | AFP-Scan (Tc-99m-labeled) | liver 7 gem-cell cancers | AFP |
| | | (radioimaging) | |
| Intracel | HumaRAD-HN (+yttrium-90) | head & neck cancer | NA |
| | HumaSPECT | colorectal imaging | NA |
| Medarex | MDX-101 (CTLA-4) | Prostate and other | CTLA-4 |
| | | cancers | |
| | MDX-210 (her-2 overexpression) | Prostate cancer | HER-2 |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath (alemtuzumab) | chronic lymphocytic | CD52 |
| | | leukemia | |
| NeoRx | CD20-streptavidin (+biotin-yttrium | Non-Hodgkins | CD20 |
| | 90) | lymphoma | |
| | Avidicin (albumin + NRLU13) | metastatic cancer | NA |
| Peregrine | Oncolym (+iodine-131) | Non-Hodgkins | HLA-DR 10 beta |
| | | lymphoma | |
| | Cotara (+iodine-131) | unresectable malignant | DNA-associated proteins |
| | | glioma | |
| Pharmacia Corporation | C215 (+staphylococcal enterotoxin | pancreatic cancer | NA |
| | MAb, lung/kidney cancer | lung & kidney cancer | NA |
| | nacolomab tafenatox (C242 + staphylococcal | colon & pancreatic | NA |
| | enterotoxin) | cancer | |
| Protein Design Labs | Nuvion | T cell malignancies | CD3 |
| | SMART M195 | AML | CD33 |
| | SMART ID10 | NHL | HLA-DR antigen |
| Titan | CEAVac | colorectal cancer, | CEA |
| | | advanced | |
| | TriGem | metastatic melanoma & | GD2-ganglioside |
| | | small cell lung cancer | |
| | TriAb | metastatic breast cancer | MUC-1 |
| Trilex | CEAVac | colorectal cancer, | CEA |
| | | advanced | |
| | TriGem | metastatic melanoma & | GD2-ganglioside |
| | | small cell lung cancer | |
| | TriAb | metastatic breast cancer | MUC-1 |
| Viventia Biotech | NovoMAb-G2 radiolabeled | Non-Hodgkins | NA |
| | | lymphoma | |
| | Monopharm C | colorectal & pancreatic | SK-1 antigen |
| | | carcinoma | |
| | GlioMAb-H (+gelonin toxin) | glioma, melanoma & | NA |
| | | neuroblastoma | |
| Xoma | Rituxan | Relapsed/refractory low- | CD20 |
| | | grade or follicular NHL | |
| | Rituxan | intermediate & high- | CD20 |
| | | grade NHL | |
| | ING-1 | adenomcarcinoma | Ep-CAM |

TABLE 8
DEMOGRAPHIC, CLINICAL AND BIOLOGICAL
CHARACTERISTICS OF THE STUDY POPULATION.

| | | |
|---|---|---|
| Mean Crohn's disease activity index (±s.d.) | 259 | (± 116) |
| Mean C-reactive protein(normalized) | 11.2 | (± 14.6) |
| (±s.d.) | | |
| NOD2/CARD15 genotype | | |
| Wild type (%) | 137 | (68.5) |
| Heterozygotes (%) | 50 | (25.0) |
| Heterozygotes and compound | 13 | (6.5) |
| heterozygotes | | |
| Female/male (%) | 122/78 | (61.0/39.0) |
| Fistulizing/refractory (%) | 58/142 | (29.0/71.0) |
| Median age (years) (interquartile range) | 34 | (25-47) |
| Disease location | | |
| Ileal (%) | 39 | (19.5) |
| Colonic (%) | 69 | (34.5) |
| Ileo-colonic (%) | 81 | (40.5) |
| Anal involvement (%) | 77 | (38.5) |
| Upper gastroinstestinal involvement (%) | 11 | (5.5) |
| Concomitant treatment | | |
| 5-Aminosalicylates (%) | 94 | (47.0) |
| Corticosteroids (%) | 86 | (43.0) |
| Azathioprine/6 mercaptopurine (%) | 111 | (55.5) |
| Methotrexate (%) | 15 | (7.5) |
| Total immunosuppressives (%) | 126 | (63.0) |
| Smoking (%) | 68 | (34.0) |

TABLE 9
CLINICAL RESPONSE TO INFLIXIMAB ACCORDING TO
FCGR3A GENOTYPE (%).

| | Complete | Partial | |
|---|---|---|---|
| Genotype | response | response | Non-responders |
| All patients (n = 200) | | | |
| V/V (n = 35) | 21 (60.0) | 8 (22.9) | 6 (17.1) |
| V/F (n = 100) | 51 (51.0) | 22. (22.0) | 27 (27.0) |
| F/F (n = 65) | 34 (52.3) | 13 (20.0) | 18 (27.7) |
| Non-fistulizing disease (n = 142) | | | |
| V/V (n = 22) | 13 (59.1) | 6 (27.3) | 3 (13.6) |
| V/F (n = 74) | 44 (59.5) | 14 (18.9) | 16 (21.6) |
| F/F/(n = 46) | 22 (47.8) | 10 (21.7) | 14 (30.5) |
| Fistulizing disease (n = 58) | | | |
| V/V (n = 13) | 8 (61.5) | 2 (15.4) | 3 (23.1) |
| V/F (n = 26) | 7 (26.9) | 8 (30.8) | 11 (42.3) |
| F/F/(n = 19) | 12 (63.2) | 3 (15.8) | 4 (21.0) |

TABLE 10
BIOLOGICAL RESPONSE TO INFLIXIMAB ACCORDING TO
FCGR3A GENOTYPE (%).

| | | | |
|---|---|---|---|
| | Complete | Partial | |
| Genotype | response | response | Non-responders |

All patients* (n = 145)

| | | | |
|---|---|---|---|
| V/V (n = 29) | 13 (44.8) | 16 (55.2) | 0 (0) |
| V/F (n = 71) | 27 (38.0) | 24 (33.8) | 20 (28.2) |
| F/F/(n = 45) | 12 (26.7) | 18 (40.0) | 15 (33.3) |

Complete response, normalization of C-reactive protein 4 week after treatment.
Partial response, decrease in C-reactive protein of at least 25% 4 weeks after treatment.
P = 0.01 (3 × 3 contingency table).
*Including 113 luminal and 32 fistulizing disease.

1. A method of improving the efficacy or treatment condition or protocol of Crohn's disease comprising the steps of:
a) determining the FCGR3A158 genotype of a subject;
b) determining C-reactive protein (CRP) levels in a subject; and
c) administering an anti-TNFα antibody to said subject if the subject is exhibits elevated levels of CRP and is homozygous for valine at position 158 of the FcγRIIIa receptor.

2. The method according to claim 1, wherein said anti-TNFα antibody is infliximab.

3. The method according to claim 1, wherein determining amino acid residue at position 158 of FcγRIIIa receptor comprises a step of sequencing the FcγRIIIa receptor gene or RNA or a portion thereof comprising the nucleotides encoding amino acid residue 158.

4. The method according to claim 1, wherein determining amino acid residue at position 158 of FcγRIIIa receptor comprises a step of amplifying the FcγRIIIa receptor gene or RNA or a portion thereof comprising the nucleotides encoding amino acid residue 158.

5. The method according to claim 1, wherein amplification is performed by polymerase chain reaction (PCR), such as PCR, RT-PCR and nested PCR.

6. The method according to claim 1, wherein determining amino acid residue at position 158 of FcγRIIIa receptor comprises a step of allele-specific restriction enzyme digestion.

7. The method according to claim 1, wherein determining amino acid residue at position 158 of FcγRIIIa receptor comprises a step of hybridization of the FcγRIIIa receptor gene or RNA or a portion thereof comprising the nucleotides encoding amino acid residue 158, with a nucleic acid probe specific for the genotype Valine or Phenylalanine.

8. The method according to claim 1, wherein determining amino acid residue at position 158 of FcγRIIIa receptor comprises:
Obtaining genomic DNA from a biological sample,
Amplifying the FcγRIIIa receptor gene or a portion thereof comprising the nucleotides encoding amino acid residue 158, and
determining amino acid residue at position 158 of said FcγRIIIa receptor gene.

9. The method according to claim 1, wherein determining amino acid residue at position 158 of FcγRIIIa receptor comprises:
Obtaining genomic DNA from a biological sample,
Amplifying the FcγRIIIa receptor gene or a portion thereof comprising the nucleotides encoding amino acid residue 158,
Introducing an allele-specific restriction site,
Digesting the nucleic acids with the enzyme specific for said restriction site and,
Analysing the digestion products, i.e., by electrophoresis, the presence of digestion products being indicative of the presence of the allele.

10. The method according to claim 1, wherein determining amino acid residue at position 158 of FcγRIIIa receptor comprises: total (or messenger) RNA extraction from cell or biological sample or biological fluid in vitro or ex vivo, optionally cDNA synthesis, (PCR) amplification with specific FCγRIIIa oligonucleotide primers, and analysis of PCR products.

11. The method according to claim 1, wherein determining amino acid residue at position 158 of FcγRIIIa receptor comprises a step of sequencing the FcγRIIIa receptor polypeptide or a portion thereof comprising amino acid residue 158.

12. The method according to claim 1, wherein the subject is a human subject.

13. The method according to claim 1, wherein the antibody is an IgG1 or an IgG3.

14. The method according to claim 1, wherein CRP levels are determined by an assay that measures the level of CRP or an assay that measures the level of nucleic acid that encodes CRP.

15. The method according to claim 14, wherein said assay measures the level of CRP and is an immunoassay.

16. The method according to claim 14, wherein said assay measures the level of nucleic acid encoding CRP.

17. The method according to claim 2, wherein determining amino acid residue at position 158 of FcγRIIIa receptor comprises a step of sequencing the FcγRIIIa receptor gene or RNA or a portion thereof comprising the nucleotides encoding amino acid residue 158.


SEQUENCE LISTING (8 sequences)

SEQ ID NO: 1 - DNA, 21 bp, Artificial Sequence
  FCGR3A specific primer
atatttacagaatggcacagg

SEQ ID NO: 2 - DNA, 20 bp, Artificial Sequence
  FCGR3A specific primer
gacttggtacccaggttgaa

SEQ ID NO: 3 - DNA, 32 bp, Artificial Sequence
  Amplification primer
atcagattcgatcctacttctgcagggggcat

SEQ ID NO: 4 - DNA, 32 bp, Artificial Sequence
  Amplification primer
acgtgctgagcttgagtgatggtgatgttcac

SEQ ID NO: 5 - DNA, 22 bp, Artificial Sequence
  Amplification sense primer
ggaaaatcccagaaattctcgc

SEQ ID NO: 6 - DNA, 27 bp, Artificial Sequence
  Amplification antisense primer
caacagcctgactacctattacgcggg

SEQ ID NO: 7 - protein, 254 aa, Artificial Sequence
  Amino acid sequence of human FCGR3A158F
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 8 - DNA, 22685 bp, Artificial Sequence
  Nucleic acid sequence of human FCGR3A158F
cagcctggctgacacagtgagacctcatctctaaaaaaaaaagcaagcagaatattttcttaaaaggcaattatattccttcttggccaggcccagtggctcacacctgtaatcccagcactttgggaggccgagatgggtggatcacctgaggtcaggagttcgagaccagcctggccaacatggcgaaaacccgtgtctactaaaaatacaaaaattagctgggcatgggggcatatgcctgtaatcccagctacttgggaggctgagacaggagaatcgcttgtacccgggaggcagagattgcagtgagccgagatcatgccactgcactccagccttggcgacagagtgaggctttgtctaaaaaaaaaaaggtattttttgcctctctgttggtaccaattgttaaattctttgtggaccactgatgcttaccaaaaaaaaaaaaaaaaaaagtgggggcatcatatttcctctagttgacattaagacacagtaatttagccagaggagatcttagcaaacatacagtccacactccactttctcatttcatgattgtagagactgagatctagacaatttaatcggtggtcaccctgggtgacatagctaggtctagagctcctggtctccaggtcagcatttctttcttcttcattaaatgtcaagtttcctcccctgttcattattagctccttccagaaagagagtttcttatctttttagtaggtactcagtaaataccaaggtattcactaggatgcctttggatgaaggtaacaagccctgacttaaattggcttaaacagcagggaaatttacttgaaattgtaagaaatctgggttggttgcgttagaagctcagtgatgtcaccaagaccatattctatccctccactctgtcctccttggctatttggcattgacctcagactggctgccttcaaaatcttaggttttgccagcagaacctaggacaaaatgagcccttgttcatgtacagtgggagagagagatcatctctcccaaacatggcactcccctctaccagattggccctatttaggacaaagttccgtcttctcccacttaaccaataaaagccaggggaatgctacaccctgagtggcttagatcagtcaagatccacctctgcatatgagggtgattcctgaatagaatcaaggttatattagaagggagggagagggatggatatcaggctagtacatcatattctatttgttgagttaactgagtcatagcaattgttgagttggaaaaaactcagaacctactgtggattcaagttcaagaaatcattctttcctacatacaacagcattgctctgtagccctgagctaagagagcatcacgaaatacagtcttcttgctgtttataatcgtaagcaaactcttggacctgggaggggatgaatggataatgtctgtctgacttgcttctttctagttagtaccaactacctcccttcttcctgtgattgttcttagaataggataaaaaatcttcccttccctagatcttacagtctccccttcccccaggcctttctatttttcaggattttactctaatcacaccaccgaagaatcaagaaatctttaaagtgtattagagtagctagttgtggcagcactaaaacacggctgcaaattctttgacactctctccatcaagaaatgaggcctacatcctctacccttgaatctgggtgggcttataacttctggtgattagactacagcagaaagagaagctgtatagcttcctaaatgttataaagtcttaaggatagctgccagcaagaccttaggacaaaatgagcccttgttcacgtccagtgggagagagagagagagaccctctctcccaaatatggcaatcctctctacctataacatttacataaatgttataggttaaacgttccacaacaaactaagtactatttaacatcaagaggaaaaagagacaggagaaagggttaataagcctgttgatgaggatctaagaagaacaaaggaggcctggtttgggcctggctacccgttggtcttgcaaagaagagtctgaggtggcagagccttcagtggcagatgccaaattatcatcatgagtgactgcaagacagtgtcagctaagatagccatttcaagctgctgaaggccttctcttttagtcgtggagtcctgtgataagaactgaaagttggaagagtgtgcttgtctgtggccttatttggtcggatgcagtctttatcatttttaatttgtttcttagaacattttatcttgttggccaaatgccctacgaaatataaaatggagtctttttctaagatggagttagttatgtcaagggtcctttatacagtcttcatcctttttcctggcatacaactcctaaaatccttagaatctccaaagtgatgtcttttggtgtgctaatgaggtaactgatggctggcagctcttaggtagcttcataacaggggctgggcacaagaaagatcatggcaaggtcagaggattggggctttcagctccaccctccaaactccctctgggaagtggagaggggctgaaggttgaattgatcaccaatagccaatgacttaattaatcattcctaagtaataaagctcccataaaaacccaaaaggacagggtttggagatcctccagagagccgaacacagagaggttcttggagggtagtgcaccagagggcatggaagctccaagccccttcccacaggtcttgccctatgtactctttacttgtgtcctttgtaatattctttatcacaaactgataaatgtaaatgtttccctgagtactgagagccactctagcaaattaattgaacccaagatgcaggtggtgggaacccccatttataactggttggtcaaaagcacaggtaaaacaacctggggcttcatcctggagtatcagaagtgtcttgtgagactgagcccttcacttgtgtcacctgatgctatttccagttagatagtgttggaattcaattgaatttgagcagaagtcccaatccccagacctgtagttgtcagtgacctcttaggaactgggctgcacagcaggaagtgaggggcaggtggggagcaaagctttatctgtatttacagtagatccccatggctcacatcaccgcctgagctcctcctcctatcagatcagctgtggcattaaattatcacaggagcatgaaccctattgtgaagtacgcatgcaagggatctaggttgcattctccttatgagaatctaatgcctgatgacctggcattgtctcccatcaccctagatgggactgtctagttgcaagaaaacaagctcggggctccccctgattctacattatggtaagttgtataattacttcattaaatattacactgtaataataatagaaatgaagtgcacaataaatgtaatgcacttgaatcatcctgaaaccatcccccacccccctacccctgtccgaggaaaaattgtcttccatgaagccaatccctggtgccaaaaattttggagaccactggattagaagacacccagttggtgcccactgctgaattgcttgcttgcttgcttgcttgcttgccagtggagagaaatccccacatatctgttgtcagaaatgtgttgtgagagcatagtgggaggaactgagtttgttttttctacagttacagcaataggtaactggaattcaactgctggactataccaaagactgccaggccagcctacctttctcacagccttcttgactacctgtcttggatgagctcactgaaagcccacataccttcattctagcatttcctcagtctggttgagctgctttggaggtaatacaggttgtaggacttccctccactcctgctcaggaccgttttcagcaggctaatcagacagcagttggcactgagtacaactggagaaatgttatcagcactgaagactgctccagctacaaatgcacagcgacccacctcagctggacctttggcattgcttggcaatccttacaactgtttggaattcttggtaattcttgtacatacaaacagtcggctccttctcccatgcaccacaatggccattctcaactctggtgttctcaaacccccaccacaactgttacccactctttctcgacaagtgtctttgactcctccttaaccaagaaaatcaggaccagcagatgtggatggtcactcaacatctgaaaatggatttgcatatgtaccccctcagctcctgccttcagctcagagcgagaggtaatccgtatccagttcacagccaactccctgtccatgtcccattcccatctcctcaggacccacacttgcttctctaggtgttccgtccctgttaggcatccaacttctcccaccctgctggcttcttcccaaagacctataaccaagctcgtatctttcacttaaaagaaaacaaaaactcccattctcttgtaactacctttgcagaattgtgtctgaaacagtgagagagatctaacttaattgactccatcttgcttctaacctccaagctgtctttcctcattcttgggcataggctgaactaactttgggagaaacttagtttatagtttgtggtttaaagcaaagatgataacagccctttcccagggcagacctcctttttttctgaagactagattgtctttgtaggactaacattagccacaagattggaaattatggtttaggaatcatgcaggtggaggctacaagattctgacccttcctaagcactgatcctaagatcggtgcttgagatattttgcagaccctgcacttgatggatcacctggcaacacccagatcaataaactggctcatctgatcttgtggtgcccacccaggaactgactcagaacaagaagacagcttcaacttcctgtgatttcatccctgaccaatcaacactcctggctcactggcttcccctccaccaaccaagttgtccttaaaaactctgctccccgaatgctctggaagactgatttgagtaataataaaactccagtctctggctcagtcagctctgcatgaattactctttctctattgcaattcccctgtcttgatgaatcagctctgtctagttaccatcctccacttctcctttcttattgtgtcacttagggctctgggttataaacaactttatcagaatccagatcttttaagtagaggaaaaagatttattggatggaaactagaggaggtgagcccatcccactgctgtgatgactgggacccaaccatctccctctcctagaagtgaatctcccttatgagtaaacaagtgtcacttattcaggattcatctcagaagagactctaatgggccaaacctcagttatatgcctgtcctctgtctgcctgtatcagttagctagcacttttataacaaagtaccacagactgggtggcttaaacaacaaaaatgtattttcttacagttcttgaggctgcaagtccaagatcaaggtgttgacagggttggtttcttttaattttttttttaaaaattttattttagatttaggggtatgcgtgcaggtttgctacataggtaaactcctatcacgggggtttgttgcacacattatttcatcacccatgtactaagcctagttactcaatagttattttttccgatcctctccctcttcctaccctccaccctcaagtaggccccagtgtgtctgttgttcttcctttgagtccatagggttggtttcttctgaggcctctctccttggcttgtaggtgtccatcttctccctgtatgggtctgtgtcccaactaacaaggacatcagtcatattggactagagcccacctaatgattttattttaacttaattacctctttaaaggctctatatccacatatagtcacatcctgaggtatagggggttccacatgtggacttcaacatatgaactgggagagacaaaatttagtccttaatagtgccccaaagtggggaaaaggaagatctggaccctcgggtttccatagtagaaagcaatcactgctttctattaagtactcacagtggggcttctccagaaagaatgatatgctaataagaaggggaggaggaagtgatcctggacagccagatgatatgtgcactattccttcataatggagattctgaagaggagaagcacttgactaaacactttttcattcctactccgtcttcaaccaaaagctgtcaaacttctgtttctcagccccagcccctgaaattgctcaggaaaaggtcattaatagttccttgattgccatatttcaatcaaactcttgtttgaattcttttctacaacattaatactgttactgttgactactccttccttgaagatctgttcccactaaacttccttgttccctcctcttcagcccctcctatacaaactcctttgtcagctatttttcctgtgcacgcttcaaaaatgtttgcatgtcaagtttctgtcattgactctctcctcttctcctctccctctcaatccctccttcctttccctcactgtttccctttaattctctctcaatactcttacagtttcagagatcttatccttactttatcttaacctaggatctctggatggattcaaatagagcttcttaaattaaaggaaacataatgtgtatatttgcatcctttcttgggagaaggcccaaaggtttttatcagaggtttgaaacctcaaccgtgttggtgcctcctaaattgtgtcttttgtcaagacctgtcttctgagttccagggccatgtgtctcactgcctactggaaatcttcacctgaaaccttcacagctacctcaaactcaataacatcaaaagctgaaatcattgtctctccctcccaaagcctgctcatcttcccattttccttttgtccatgaaagctactgccatcctccttatcacccaaattagaaatccgagcatcacccagacctctcccccttcatcacccctcagccaatcactcaccaagtcttgtccatccttccttcctaacttctctcctggatgcttccattgcatatccactttttaaacagagtggctcttgtctcaactagactgttgaaataatcttccaacttttccctccaccttccatctctctcccctctaactcattccttggactgctgtcagagtacttttcataaaatataaaacagatcttgtgattccccagtctaaagcctttttattagttcccattaccttttagaataaaatatgtactgttcatcctgacacacaaaactcttcgtgataaatactaattgagtgcctagtatgtgcctgcccttgtgctaaatgttgagggtacaggggtaaacaaggtgaacagcttccctgctctccaagacctttcagtccacaaatgcaatgagtttacagaggagaagcacaagctcctaaaggagttggggtggggttgggggtcagaacctaatttagaaaattgaggaaggtctcaacctcccatcttgcatttacaatagtaatcagcaggtgtggtaccaaatatggaaccaacaattttatctgcattatctcatttaagccatgagtgccattattgttagcctcactttacagataaggaaactgagggctagaaggttaaataagtggcagagttgggatttcctccagattcctgtgagacccagacatcttaatccttttggaacctgtgcttctcctttgtagtactcactacacttgtggaactacatccaactacacttgtggaactacagccagctctgcaaacatgacagtctacttcactccaagtctttgctcatgctgctcctcttgcctggaatgcctatttctctcaaaaatcttcctgctgaatattttgcgatctaattaaagtgttctctcttccatgtacactcctccctcagatagaattagccactgtcttctttgtgcatacacagcatttcataaatactgtcacagtccctctagcacttcaaatacttatctgatgttctccccctaagaaactgtaagtcctagaggatgacaatcaactgaattccatagtcagaaacttctgctgtgcctggccttccaatgagaaaaggagagaagaggaggggaaggaagaaaaagggaaggagaagaaagaaaagcaaacatgaagataaacacttcaatatatgatatcccaagaccatctacccttttgtaaaaattttgctttttttttttcccccccaagagtcagggtctcactctgtcgcccaggctagagtgcagtgccatgaacataactcactgtagcctctaactccggggctcaagcaatcctcctgcctcagcctcctgggtagctgggactacaggcatgcaccaccacatctggctattattattattactatattagtagagatggggtctttctatgttgcctaggctggtctcaaattcctggcctcaagcaattcttccacctcacattggccttccaaagtgctgggattacaataagccaccataggccaaaattttgcattttatccattactgtaaaattaacccttagaaatccaacaacactcaatttgagaattgttcaacaaccacttaatgaaaaccccctgaaagcttcccatcctgttgcagtccctttctctcctcctgtgctctctcctcttcttcctatctagcccacccttttggcagctaagaattcctccctccattggagagccacagaccaaagaggagtcaaataagaaaataagacctcaaagaaggaaaacaaagtgaaggccttgcatcagaagtcacgtggcagaaagccacctggatatctgaaaagaagaaagaattgagggatatccgctttttgcctcagagaccatccttagccctgaaggctttgtttctgctttaggtttcccagataagcatccgaagtgctacagcaaggaactttaagtttccagatacttgtctggattttgcaaggcgtagatgagtcacttgagaaggagaactggaatggctgcctgggttcatttccattgtccaatccaagggcctgtggagaaggggctgctgcaagactctgtgtgtggcggggggaggggtgggtacgtggatggcaatgggaggatcaattaactccacccaggagccaaatgaaacacacaaataaaaaacaaaacctgagtagtggtttttaggtcattctggagtagaaagagcattcatttatagcaaaggttggcgggcacctgtgtcagcccctgcctccactccacccctaacaagtatcaggtgcccacacgggcctgctgctcgcctcctgggcttttctaagccaggtgagacctgtcccagatgtccacgaatccactgggggagtggcactatcaagcagagtcatctgattttctgcctgggacctggaccattgtgagagtaaccaacgtggggttacgggggagaatctggagagaagagaagaggttaacaaccctcccacttcctggccacccccctccaccttttctggtaaggagccctggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcaggggctggggaaaggctgtttacttcctcctgtctagtcggtttggtccctttagggctccggatatctttggtgacttgtccactccagtgtggcatcatgtggcagctgctcctcccaactgctctgctacttctaggtaagtcagggtctccctggttgagggagaagtttgagatgccttgggttcagcagagaccccttttcaggctacgaatgagactcccacgaagggatgggacccctcaccacatctatagctgtggattgagctcctaggacaagccaagatggggctagaaatgaggagaatgctggttccaattggggcatactcatgagtgaggccagtcacttcacccctctgggtcccagaatcactctgtggaaccaaagagcttcgactagatggtccctagggtctgtctctttcagtttgacattccagggttctcctctatgattttcaatttctaccctttcttgtggggatatgggttgaggctctttctgtagcttggttcagggaaattcaacctgtacccttaatttgtgagtttgcacagggagcaaggggtaagggagcagtgttgaaaatagggatttgtgttgacagtggcgcaagaggcatgaacagtggagaccagagagcaggtagcaaggtttccaccagaaacatcctgattcttgggaaaattgggctcctggggcagaggagggcaggggagttttaaactcactctatgttctaatcactctgatctctgcccctactcaatatttgatttatcttttttcttgcagtttcagctggcatgcggactggtgagtcagcttcatggtcttggattgacccagtggggcacatatggggacaaaggccataagatattgggaaatgcttgttgaatgggaaaatgctgatgtggggttagcagggatagttcctccaacacagcagaacttggccctgtgcttctctggccagctttccttaagatactgaacaggccaaaaatggggccaagatgctctaagactgagccaccaagcatgggtttgcaatgagctcattctggctttgaggctccctgggaatggcagtgtagagcctgctcctctccctgtcctcaccccacattatcttggctcctcagaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggtgagttgatgaaggggaagaggaaaatcaccaataaagggtgaaacaaagggtcctgaaatacttggtaagagccagagatgatattcttagagataaaagctaagatgagatgatgtgtggtcccactgaatggtatcagagttgtagtcctagctctaagtaggtcttgggcaaaatgtcaaagcctgtcagacagtagatataggactgctgcattgcacaattccaagaatccccatatggagtgcatacaatgtgaatgtgtcatgtgaaggttaggccatggcatagatgctcaataatagttatttatatatttattttcattttttttaattttattttttgagacagagtatcactctgtcacccaggctggagtgcaatgcggcaatctcagctcactgcaacttctgcccccttgggttgtagtgattctcctgcctcagcctcccgagtagctgagattacaggcacccgccaccacgcccagctaatttttgtatttttagtagagacagggtttcaccatgttggtcagtctggtctcaaactcctgacctcaggtgattcaccagccttggcttcccaaagtgctgggactacaggcgtgagccaccacacctggccaataatatttattgaataaattaatgaatttggtgttaggacctcaatctccttctcgctctcagacatgtaatgccctaagccacctcccaaagcaatcctagtggcctagcatcatatctttctgtctcctcatcaatgctatactcaaacctataattaagcataaatttggtaatgtgatagctcttccaatagaggcagatacatgttcagcctgcacattaatcatgacatgaaagttcttgtgtactattaacagaatatagacgtcagacacaggtaggagaaatattttgaaggcagaggtctttcctggtgtccctacaatcttaccacataggctggtccctgcagtgtcgccctgcaaacctaactctacttccacggctgttccattcatacaatgtttatgggtggaacaagctttgggggaagaagggcataaggaggtggatctgcaagagagctccatggaattgggcctctgaaactgatttttgtggctctttggcctctgacagtaccactcaactgacatggtcttcactctccagagctacaagaagatatgtccatttctagctaggtaagagatgtccacctacaaccaaataaaatgggggaattaccaagagaaagcaatagaaaaatcaagtctaagagttactagtttgccttgaacttggctctagaaactggctttagaagtctagccaatcaaggctatattaaactgtgaccatgagaattagcttcaccaggtaaacttctgagcatcctttaatcctttaggacccatttcacttatgtcctcctctgagaagcattttttacttctttttttgtttgtttgtttgtgtttgtttttgtttttgtttttgagacagagtctctctctgtcacccaagctggagtgcagtggcgcaatcttggctcactgcaacctccacctcccgggttcaagcaattctcctgcctcagcctcccaagtagctgggactacaggtgcatgccaccacgcccggctaattttttgtatttttagtagagacagggtttcgcagcgttagccaggatggtcttgatctcctgacttcatgatctgcccacctcggcctcccaaagtactaggattacagatgtgagccaccgcgcccagcctgcattttttacttctttcaggcagaatttctttattccaatctagtcagccccgcagtcctttattcttagcctgttgtagcacttgtcatattgtattgtgattatttctgaatatttatgtttctatgtctagactgtagattctttgaggctgagaactatatgtcccatcatctgggtatctccagtccacagtgtgtcatacatagtgagtgcttgatgaaatatcacttgaaggaatatacatatggacattcactgggtccatgacaggatagattcgaacaagaatgttcctccaaaggccaccagactatatactaaccatgactttatgctaataatgattcatctctctgctgaaaaagtaagtggatagataggcacatggcttcttttgataaatgatatctcttaataggtaatgaagattactttctgtttggcaaatctttgtggtagagaatcatgaccaacacacgtcctaccaattttgtttagcatcaggtagtagattttttaaattatagtaattcaagctgagaatgtagatttaaaaaataaaattattgtaaattttgttttgttcttattacaaaagtcatttggggtcaatttcaaaaatatataaaagtaaacaggagaaatttaaaatgtccttcagtcccactccttcagagaaaacccctgttaatatgtaagtgcatatccttcttttttctgtgcataatactttttaaaatatttgaagtattatgcttttttaacttaaaattgtctcatgaatattttcttatgccattataatacttacctataacatcattattttttaattattcaggccctttcccgaccatgacctcatgttctctctttgtgaagtctgattacttggtgacatgatcgtgagaataagctctggcgatataagaatttcctctcttgaaggccatgctcagtaaattacttggtgacatgatcgtgagaataagctctggcgatacaagaatttcctctcttgaaggccatgctcagtaataaagttggtctcaccgaggccctgtgacaccttagaaaccacgaattgccaggctgagcaataccagtcccgcccttcccctccctggtgtttacattgagttctccttcacaatttctgcagccactccgtggccaccgtcaccttattcctgactgccacaagagtctttcaatattcctttgattgcctattccttctgaaatctaccttttcctctaatagggcaattcatcattttcaaatgcaatttttactctgatctagaacttactgtgaatccttgtcacctgccacagcaaatctaagtctagcacttaaggatcctgcagatatgctcatcgttgcttctcacttacctcattgcttagtccctctgctctaaccctgtgtgttgatcacatgtgtgtgtgtccctcttccccattagacaaaggtcttggtatgacttcagttctcttgcagggccccatcagctcttccccaaagggagctatgcagggttgactcccaatctggctttcccttatgtctcaggatctgggtggtacgtggccccttcacaaagctctgcactgagagctgaggcctcccgggcctggggtgtctgtgtctttcaggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtgagacatgtgccaccctggaatgcccagggacgcctgtgtgtggaacctgcaatcacactgggaagttgagttgggaggagattcctgattcttacacgcacttcttcatatgtggttccctcctggtgatcaccaggaggtccccaaaagtccctgattgcagggtaggtttgcagctctgtttcagtccattcttttggggtagctaggaggtgtcattcactctgcagcatgatggcaggagcagaagccacatctcctccccaataaatacctctgtctttccttacgctaatcacacccacggtgtcatatgttcctatcgtgctggcctccttcttatccaagccttttagccacgatccaaactggcaggagcccctcatcccctcacagaaagagcccagaacctgggttctggccctgcagctaattaaccatctgaccagaggtgagccacttagtctctctgaaccccaatttcttcttccgtaacaaaaataagctgacatttattgggcacctttcagtgtgctagactctgtgctaaacaattctttacatgcacctggtttgactatcacagtagaccttcacaacatgagataggtaatattccattttacagatgaagtaaccgaggtgcaaaaataaataaataagtttccctaaggtcacatcaaagacttcaaagcctgtatatttaaccagtaagtaaaagatttgaacaagcactaatatcctatgatcccattaagtcatccacaaaacatctctaggttctgtagcaccagcctccagaatcagagctctagagtggtgtgcctggactttccagtttcacagaacttctatctgtaactagcccaagacataaattgtaaacaatttgcatgtagaaaggcagcaaaacaccttttgagattttgacactacaatgccataatttgtacaaaaataatttcatgacactttaaactgaaagtaaatactcccaagtggttagggaaagagagcaaataaagcaaatggggtaacatgtaaacaatgagtggatctgggtaaaggatatacgagattaaactattctggtcattttttttttaagtttggaaatatatcaaaatcaagagtttaaaaaattgaaatgcaaaatcaacaaatttgtcccagtttctagaccatagcattgtctgacaatttcttaactgtcacacaaaacccagcttacaacctaacttgttaacgctccctgtcacatctctgtcaaacaagcaggagcctttgctcagtgtttggtgagctgtcctctgctcagatagcactaagatcaggaaccaatgggaggaagcaatactttcccccagacttccccaccattcctaccacttgcctgttggctgttgtcaaagactttctactggtgacctcactgtttgttccaaatatctgccttagtgactgtcattttttttcatctctccacttctcctaataggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaattctttcttcttccctttgtccaacattgtaacagagattggtttggattggtaagaaacaccccctcctcccagcaaccatctcaccacaactcatataaattagccagcttgctttccaaatcttgctgagacaattgggctaaggaggattcttatgggaagtatgggataggagggtgaataagcattagagatcgttttagagcattggggcagataggagaaggcacagctacacaggaggtagaggcctgggcagaggtagagggtcagcctgattgtatgaattatgagctatataccaagacgattcaagctagattgcatacataaatattacataagattccgacacgacacaggtgcatttggaaaccttggacattcaactcacatttatttactacctacaatgtgcaagcttgagttcaggtgctgaagataccagatgaacaacacagggtcattccctggagaagctttatttctagtgagaaaaacagttaaataggaagagaatgaagaaagggctgcagaaaagaggcttgatttggggggtgtggtcatgaaggatgagtaggagttcgccaggcaaagaagagaagaaaagcccaaggttcataggcaaagattcaaaaaccagagtgtgagttcaagaaagcagtttggttctgtgtcggtgagggagaggaaagagtttcagggccagatcatgaagggcattaccttccaaactaaggagatcgtatcagaccctgcaatacattgagagagtttaagcagaccaggtttgtaccgtatagtattttagaaggattctctcgcaactacttgatggatggacgggacaggagagttgaagaccagaagccaaatagggcagcaaggcaggatgcagtaacccaaagggagcaatgaggaagtaactggcggtgaggctggagaggaaggtgcttaatcaacaaggtatttaggaggccgactctccaagaattggcagccagcagtacacggcgtgactaaggaccaggttccacacatagtgcccgttttctgagttaggaaatagaaaggcaaggcaggtacaggtttggtggaaagacaaacaattcgttttggtattattagtacttacttcctttggtcagtaaattttcttaaagtgtcagtttccataacgtaattgccgtggttaagcagctaagagttatcactacaaccctagtcggaaaaaccaaatacctcaaaattacccgtacagcactaaggcagaagaggacattgggaaccacacaacgcggaggtctgctaccagagctccctgcggttagcaccgcggctggttttgagcgccaaggccccagcgctcccagcggatagcatcgcacgcagttttttcagtcaaagtttcaaaaacccagggttcacaaaatgcgacttccgtccctgggtgggatcgaaccaccaacctttcggttaacagccgaacgcgctaaccgattgcgccacagagacgggcgttggcgattttggctgccaagtcacttcactgaagaaaaaatgctcagcactcacgtctccaaaaaaattgaggttgatttgaaaccagtgacacaattagctttccgtgcttcagggcgcggctcatagccctgagcgaggcaggtcttttttctgcgctagcacttgcctagatctggagcaggactcagcttccagcagaagaggttgagaaaaggagagcagaagagaatgcaggaacgaagggtcttcggggaatccaaaatggatgctctctgtgggttcgggggttccgttgattttggtcagagaagtacgacgataagctttttttgctgatgtagacaacttatgtatgcatgtgcacacgtttagtgctgactcataataagcttattatcgtgagcattaaaaatattttctttcaggtccaatcacgtccagcaaaatgtgatgtctaagtaagtgagttttgtgttacaaaattagtcttcaacccacgctgttttgaaaggtttctaccggcatattagacatgcagacagaacacggagcttaaaaagcctgtaacattccaattaatggtattcagcttggaaataaaaaatattttttaaaaaatgcgtgcaacttaaggactttcatgctgacatatccagatccaaatatctgaggacagagacccctaattccaccaccatcgacctagggaacctcgtcagtgctgggtctaaaaaggctttttttttttctttaattcatatgtatatatactttattcatatatatatatactttaagttctaggctacatgtgcacaaggtgcaggttcgttacatatacatgtgccatgttggtgtgctgcacccattaactcgtcatctacattaggtgtttctcctaatgttatccctccctccttcccccacccacgacaggtcccggtgtgtgatgttccctaccatgcacacgtatgtttattgtggaactattcacaatagcaaagacttggaaccaacccaaatgtccatcaatgatagactggattaagaaaatgtggcacatatacactatggaatactatgcagccataaaaaaggatgagttcatgtcctttgtagggacatagatgaagctggaaaccatcagtctgaacaaactatcacaaggacagaaaatcagacaccgcatgttctcactcataggtgggaattgaacaataagaacacttggacgaaagccattttctatattgcccaaaaaccagggtctctccatagcctccacacagaatctcctttctttctgccctgccatcctctgtcatcagtgggctccagtttaggagcaggtggaagttttcaatgatgttcagtgaaatgagaagacatgcaaacatagatatgtatatgcagaaattatatatgcatatatgtttatatgtacacagtatcatatgtataataaataagtaaataaataaataaatttgccaaatgatctttaaactagagtcatttattttttttattaatttttttttttgagatggagtcttgctctgtcgcccaggctggagtgcagtggcgcaatcttggctcactgcaacctccacctcccacattcaagcaattctcctgtctcagcctcctgagtagctgagattacagtcatgggccaccatgcccggctaatttttgtatttttttttttttttttttgagacagagtctcgctgtcgcccaggttggagtgcagtggcgcgatctcggctcactgcaggctccgccccccgggattcacgccattctcctgcctcagcctcctgagtagctgggactacaggcacctgccacctcgcccggctaatttttttgtatttttggtagagatgggctttcgctatgttggccaggctggtctcaaactcctgacctcaggtgatcctcctgcctcagcctcccaaactgctgggattacaggtgagagccactgtaccaggcctagagtcatttcttttatactttaaatttttgtctctgttcttttgctcagacctgtggagctggcaatatgggcaagtgtcatggactgtctactgccaggaagctccattgtcaccgacaggatcagaagtggcatggtaaatggtacaagaaagcccattcgggcacagtcctgaagaccagcctttttggaggtgcttctcatgcaaagggaattgggctggaaaaagtagggattggagccaaatagcccagctctgccactgagaagtgtgccagggccaagctgatcatccagcataagctagatgctgtggtctccactggcacagctgatcctcttgttacaggatggaggctgtgaggcagatgagagaacagcaagaaaatcacagcctttgtacctgatgatgattgcttgaattttattgaaaaaaatgatgaagttctgtatcaggggaaccagcacccaatatttcaatgtaggttct